(19) United States
(12) Patent Application Publication
(10) Pub. No.: US 2005/0214796 A1
(43) Pub. Date: Sep. 29, 2005

(54) Compositions, methods and detection technologies for reiterative oligonucleotide synthesis

(76) Inventor: Michelle M. Hanna, Phoenix, AZ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/976,240

(22) Filed: Oct. 29, 2004

Related U.S. Application Data
(60) Provisional application No. 60/514,908, filed on Oct. 29, 2003.

(51) Int. Cl. C12Q 1/68; C12P 19/34

(52) U.S. Cl. 435/6; 435/91.2

(57) ABSTRACT

The present invention provides methods for detecting the presence of a molecule of interest by generating multiple detectable oligonucleotides through reiterative enzymatic oligonucleotide synthesis events on a polynucleotide sequence and detecting said products. The invention also provides for methods of multiplex abortive transcription. In a further aspect, the invention provides for the use of dendrimers containing abortive promoter cassettes. In one aspect, the invention provides for abortive promoter cassettes suitable for use in the present invention. In one aspect the products of abortive transcription are detected with the use of mass spectrometry. In another aspect, the invention provides a method for detecting a target protein, DNA or RNA by generating multiple detectable RNA oligoribonucleotides by abortive transcription that are detected, including by mass spectrometry.

### CROSS REFERENCE TO RELATED APPLICATIONS

This application claims the benefit of the filing date of U.S. Provisional Application Ser. No. 60/514,908, filed Oct. 29, 2003, which is incorporated herein by reference in its entirety.

### BACKGROUND OF THE INVENTION

1. Field of the Invention

The present invention relates generally to the detection of molecules of interest by generating multiple copies of oligonucleotides through reiterative synthesis on a nucleic acid template, via abortive transcription initiation. The multiple copies of oligonucleotides are detected via methods including mass spectrometry, indicating the presence and/or particular characteristics of the target molecule. The methods of the invention may be used to detect pathogens, toxins, proteins, nucleic acids, mutations, cancerous conditions, or other molecules, diseases or conditions.

2. Related Art

The development of various methods for detection of nucleic acid amplification products has led to advances in the detection, identification, and quantification of nucleic acid sequences in recent years. Nucleic acid detection is potentially useful for both qualitative and quantitative analyses. For example, nucleic acid detection may be used to detect and identify pathogens; detect genetic and epigenetic alterations that are linked to defined phenotypes; diagnose genetic diseases or the genetic susceptibility to a particular disease; assess gene expression during development, disease, and/or in response to defined stimuli, including drugs; as well as generally foster advancements in the art by providing research scientists with additional means to study the molecular and biochemical mechanisms that underpin cellular activity.

A wide variety of nucleic acid detection technologies have been developed, several of which have been advanced as suitable means for detecting the presence of low levels of a target nucleic acid in a test sample. One category of such methods is generally referred to as target amplification, which generates multiple copies of the target sequence, and these copies are then subject to further analysis, such as by gel electrophoresis, for example. Other methods generate multiple products from a hybridized probe, or probes, by, for example, cleaving the hybridized probe to form multiple products or ligating adjacent probes to form a unique, hybridization-dependent product. Still other methods amplify signals generated by the hybridization event, such as a method based upon the hybridization of branched DNA probes that have a target sequence binding domain and a labeled reporting sequence binding domain.

There are many methods of target nucleic acid amplification known in the art, including isothermal target nucleic acid amplification, which have been developed to meet the demands for rapid and accurate detection of pathogens, such as bacteria, viruses, and fungi, for example, as well as the detection of normal and abnormal genes. While all of these techniques offer powerful tools for the detection and identification of minute amounts of a target nucleic acid in a sample, they all suffer from various problems.

Accordingly, there is a need for rapid, sensitive, and standardized nucleic acid signal detection methods that can detect low levels of a target molecule.

These needs, as well as others, are met by the inventions of this application. The methods of abortive transcription have been described in International Patent Application PCT/US02/34419, filed Oct. 29, 2002, published May 8, 2003 as WO 03/038042; U.S. patent application Ser. No. 09/984,664, filed Oct. 30, 2001, published May 29, 2003 as U.S. Application Publication No. US-2003-0099950; and pending U.S. patent application Ser. No. 10/425,037, filed Apr. 29, 2003; published Mar. 18, 2004 as U.S. Application Publication No. US-2004-0054162-A1; all of which are incorporated herein by reference in their entirety.

Abortive transcription offers several advantages over other methods. The method is relatively simple; involving few steps, may be performed isothermally, and with inexpensive reagents; is rapid; highly sensitive; and readily adapted to different conditions. The nucleic acid being amplified need not be the actual target molecule of interest. Rather, the amplified nucleic acid may represent, for example, the presence of a protein, nucleic acid or other target molecule of interest as detected using steps that ultimately involve nucleic acid amplification via abortive transcription.

The invention provides methods for detecting the presence of a target molecule (such as nucleic acid sequence or protein) through reiterative synthesis events on a nucleic acid template by generating multiple oligonucleotide transcripts. The multiple oligonucleotide transcripts may be detected via a variety of means, including FRET, radioisotopically, colorimetrically, enzymatically, and mass spectrometrically. Mass spectrometry can detect a molecule that has not been “labelled,” is rapid, sensistive, and accurate.

In general, mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). Mass spectrometry has been used for analysis and characterization of organic molecules by the determination of the mass of the parent molecular ion. In addition, by arranging collisions of this parent molecular ion with other particles (e.g. argon atoms), the molecular ion is fragmented forming secondary ions by the so-called collision induced dissociation (CID). The fragmentation pattern/pathway very often allows the derivation of detailed structural information. Mass spectrometric methods can be found summarized in Methods of Enzymology, Vol. 193:“Mass Spectrometry” (J. A. McCloskey, editor), 1990, Academic Press, New York.

Due to the analytical advantages of mass spectrometry in providing high detection sensitivity, accuracy of mass measurements, detailed structural information by CID in conjunction with an MS/MS configuration and speed, as well as on-line data transfer to a computer, there has been considerable interest in the use of mass spectrometry for the structural analysis of nucleic acids. Recent reviews summarizing this field include K. H. Schram, “Mass Spectrometry of Nucleic Acid Components, Biomedical Applications of Mass Spectrometry” 34, 203-287 (1990); and P. F. Crain, “Mass Spectrometric Techniques in Nucleic Acid Research,” Mass Spectrometry Reviews 9, 505-554 (1990), and U.S. Pat. No 6,602,662.

Mass spectrometric detection has generally been limited to low molecular weight synthetic oligonucleotides by determining the mass of the parent molecular ion and through this, confirming the already known oligonucleotide sequence, or alternatively, confirming the known sequence through the generation of secondary ions (fragment ions) via CID in the MS/MS configuration utilizing, in particular, for the ionization and volatilization, the method of fast atomic bombardment (FAB mass spectrometry) or plasma desorption (PD mass spectrometry). As an example, the application of FAB to the analysis of protected dimeric blocks for chemical synthesis of oligodeoxynucleotides has been described (Wolter et al. Biomedical Environmental Mass Spectrometry 14, 111-116 (1987)).

Two more recent ionizations/desorption techniques are electrospray/ionspray (ES) and matrix-assisted laser desorption/ionization (MALDI).

Electrospray ionisation and matrix assisted laser desorption ionisation mass spectrometry are “soft” ionisation techniques whereby samples are analysed to produce primarily molecular weight information.

Electrospray ionisation is equally suitable for the mass measurement of most small organic molecules as well as proteins and other high molecular mass biomolecules. In general, positive ionisation electrospray is used for the analysis of peptides, proteins, glycoproteins and small molecules containing amines and other functional groups capable of holding a positive charge. Negative ionisation electrospray is used for the analysis of oligonucleotides, saccharides, and small organic molecules containing acidic functionalities and other groups capable of holding a negative charge.

With electrospray ionisation, the sample is introduced in solution to the mass spectrometer and so the technique can be coupled directly with HPLC or CZE to generate on-line chromatographic data providing molecular weight information for all the components, thus avoiding the need for off-line separation followed by isolation and analysis of the individual fractions. For further discussion of ES/EIS mass spectrometry see, e.g. Yamashita et al. (J. Phys. Chem. 88, 4451-59 (1984); PCT Application No. WO 90/14148). Current applications are summarized in recent review articles e.g. R. D. Smith et al., Anal. Chem. 62, 882-89 (1990) and B. Ardrey, Electrospray Mass Spectrometry, Spectroscopy Europe 4, 10-18 (1992). The molecular weights of a tetradecanucleotide (Covey et al. “The Determination of Protein, Oligonucleotide and Peptide Molecular Weights by lonspray Mass Spectrometry, Rapid Communications in Mass Spectrometry, 2, 249-256 (1988)), and of a 21-mer (Methods in Enzymology, 193, “Mass Spectrometry” (McCloskey, editor), p. 425, 1990, Academic Press, New York) have been been determined. As a mass analyzer, a quadrupole is most frequently used. The determination of molecular weights in femtomole amounts of sample is very accurate due to the presence of multiple ion peaks which all could be used for the mass calculation.

MALDI mass spectrometry, in contrast, can be particularly attractive when a time-of-flight (TOF) configuration is used as a mass analyzer. The MALDI-TOF mass spectrometry has been introduced by Hillenkamp et al. (“Matrix Assisted UV-Laser Desorption/lonization: A New Approach to Mass Spectrometry of Large Biomolecules,” Biological Mass Spectrometry (Burlingame and McCloskey, editors), Elsevier Science Publishers, Amsterdam, pp. 49-60, 1990). Since, in most cases, no multiple molecular ion peaks are produced with this technique, the mass spectra, in principle, look simpler compared to ES mass spectrometry.

Various mass spectrometry techniques have been used to analyze DNA of different sizes (Nelson et al., “Volatilization of High Molecular Weight DNA by Pulsed Laser Ablation of Frozen Aqueous Solutions, Science, 246, 1585-87 (1989); Huth-Fehre et al., Rapid Communications in Mass Spectrometry, 6, 209-13 (1992); K. Tang et al., Rapid Communications in Mass Spectrometry, 8, 727-730 (1994); Williams et al., “Time-of Flight Mass Spectrometry of Nucleic Acids by Laser Ablation and Ionization from a Frozen Aqueous Matrix,” Rapid Communications in Mass Spectrometry, 4, 348-351 (1990)). Japanese Patent No. 59-131909 describes an instrument, which detects nucleic acid fragments separated either by electrophoresis, liquid chromatography or high speed gel filtration. Mass spectrometric detection is achieved by incorporating into the nucleic acids, atoms which normally do not occur in DNA such as S, Br, I or Ag, Au, Pt, Os, Hg.

All documents or publications cited or identified in this application are hereby incorporated by reference in their entirety to the same extent as if each individual document was specifically and individually indicated to be incorporated by reference in its entirety.

### SUMMARY OF THE INVENTION

The invention provides methods and compositions for producing multiple detectable signals through reiterative oligonucleotide synthesis reactions on a polynucleotide template for the detection of target molecules, said signals being reiterative oligonucleotide transcripts that are detected through the use of mass spectrometry. The invention also provides applications for the reiterative synthesis and detection methods. Applications of the methods and kits of the invention include, but are not limited to, detection of mutations and single nucleotide polymorphisms, RNA molecules, pathogens, and detection of pre-cancerous or cancerous mutations and conditions.

In one aspect, the invention provides a method for detecting multiple reiterated oligonucleotides from a DNA or RNA polynucleotide. The template from which multiple reiterated oligonucleotide transcripts are produced may or may not be the target molecule. The method comprises: (a) incubating a single stranded target polynucleotide with an initiator, a polymerase, and optionally additional ribonucleotides and optionally a target site probe and optionally a chain terminating nucleotide; (b) synthesizing multiple oligonucleotides from said target polynucleotide, wherein said initiator is extended until said terminator is incorporated into said oligonucleotides thereby synthesizing multiple reiterative oligonucleotides; and (c) detecting or quantifying said reiterative oligonucleotide transcripts of a polynucleotide of interest with mass spectrometry; wherein said oligonucleotides being synthesized are of lengths selected from the group of lengths consisting of: about 2 to about 26 nucleotides, about 26 to about 50 nucleotides and about 50 nucleotides to about 100 nucleotides, and greater than 100 nucleotides.

Further modifications of the above methods have been described in International Application WO 03/038042, published May 8, 2003 and U.S. Application Publication Number US-2003-0099950, published May 29, 2003, both of which are incorporated herein by reference in their entirety. Such modifications include, but are not limited to: the use of additional steps, such as deaminating a single-stranded target DNA sequence under conditions which convert unmethylated cytosine residues to uracil residues while not converting methylated cytosine residues to uracil; the use of capture probes or immobilizing sequences; modifications of target site probes, initiator and the like to detect single nucleotide polymorphisms and changes in methylation; the use of abortive promoter cassettes; the use of multiple target site probes, initiators and the like; the use of different reaction conditions, polymerases, nucleotides, labels and the like; modified nucleotides, nucleosides; and kits of the invention.

In one embodiment, the invention comprises a method for detecting the presence of a target molecule comprising: (a) synthesizing multiple copies of oligonucleotides through abortive reiterative synthesis on a nucleic acid template; and (b) determining the presence of said target molecule by mass spectrometry, thereby detecting the presence of said target molecule. In another embodiment, the molecular mass and/or abundance of said multiple copies of oligonucleotides is also determined.

In one embodiment, the target molecule comprises the nucleic acid template. In another embodiment, the target molecule is distinct from said nucleic acid template. Abortive transcription may also occur from an abortive promoter cassette.

In one embodiment, the invention comprises abortive promoter cassettes. Descriptions of a genus of certain abortive promoter cassettes and specific embodiment are also provided. In related embodiments, the invention comprises methods using such abortive cassettes for abortive transcription and related methods.

Such abortive promoter cassettes may, in some embodiments, be linked to a second molecule with specificity for the target molecule, said second molecule selected from the group consisting of: a DNA sequence; an RNA sequence; a PNA (protein nucleic acid) sequence; an antibody; a binding protein; a signalling molecule; a hapten; biotin; a binding nucleoside; a binding nucleotide; an enzyme substrate; an enzyme substrate derivative; and an ion.

In one embodiment, the invention comprises a method for detecting the presence of a target molecule comprising: incubating a template polynucleotide with an initiator and a polymerase; synthesizing multiple oligonucleotides from said template polynucleotide, wherein said initiator is extended until the transcript is terminated, causing multiple reiterative oligonucleotide transcripts to be synthesized; determining, by mass spectrometry, the molecular mass and/or abundance of said multiple reiterative oligonucleotide transcripts, thereby detecting the presence of said target polynucleotide.

In some embodiments of the methods of the invention multiple reiterative oligonucleotide transcripts further comprise a modified nucleotide. In other embodiments, the method further comprises incubating said target polynucleotide with an initiator, a polymerase, a terminator and a target site probe. Such target site probe may be of a size selected from the group consisting of, but not excluding: about 20 to about 50 nucleotides; about 51 to about 75 nucleotides; about 75 to about 100 nucleotides; and greater than 100 nucleotides.

In some embodiments, the target polynucleotide is DNA, and the multiple reiterative oligonucleotide transcripts comprise RNA.

The nature of the target molecule may also vary. In some embodiments, the method comprises a target molecule selected from the group consisting of: a protein; nucleic acid; RNA; DNA; a Carbohydrate; a Lipid; an Antigen; a Hapten; and an Ion.

In some embodiments, the invention provides a method for detecting a pathogen. In other embodiments, the method comprises a method of detecting a molecule from an organism selected from the group consisting: HIV-1; HIV-2; HIV-LP; polio virus; hepatitis A virus; entero virus; human coxsackie viruses, rhinovirus; echovirus; Calciviridae; Togaviridae; equine encephalitis virus; rubella virus; Flaviridae; dengue virus; encephalitis virus, yellow fever virus; Coronaviridae; SARS; Rhabdoviridae; vesicular stomatitis virus; rabies virus; Filoviridae; ebola virus; Paramyxoviridae; parainfluenza virus, mumps virus, measles virus, respiratory syncytial virus; Orthomyxoviridae; influenza virus; Bunyaviridae; Hantaan virus; bunya virus, phlebovirus; Nairo virus; Arenaviridae; Reoviridae; reovirus; orbivirus; rotavirus; Birnaviridae; Hepadnaviridae; Hepatitis B virus; Parvoviridae; Papovaviridae; papilloma virus; polyoma virus; Adenoviridae; Herpesviridae; HSV 1; HSV 2; varicella zoster virus; cytomegalovirus (CMV); Poxviridae; variola virus; vaccinia virus; Iridoviridae; African swine fever virus; Hepatitis C; Norwalk virus Helicobacter pylori, Borelia burgdorferi, Legionella pneumophilia, M. tuberculosis, M. avium, M. intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Actinomyces israelli; Cryptococcus neoformans, Histoplasma capsulatum, Coccidioides immitis, Blastomyces dermatitidis, Chlamydia trachomatis, Candida albicans; Plasmodium falciparum; and Toxoplasma gondii. Such molecules could be DNA, RNA, proteins, toxins, carbohydrates, lipids, antigens, or haptens, for example.

The invention also includes methods for detecting cancer, DNA methylation, a mutation, a disease comprising: (a) synthesizing multiple copies of oligonucleotides through abortive reiterative synthesis on a nucleic acid template; and (b) determining the presence of said target molecule by mass spectrometry, thereby detecting the presence of said target molecule. In another embodiment, the molecular mass and abundance of said multiple copies of oligonucleotides is also determined.

The invention also includes methods for detecting and quantifying RNA expression.

A variety of mass spectrometry methods be may used according to the methods of the invention, including (but not limited to) fast atomic bombardment (FAB) mass spectrometry, plasma desorption (PD) mass spectrometry, electrospray/ionspray (ES) mass spectrometry, matrix-assisted laser desorption/ionization (MALDI) mass spectrometry, and matrix-assisted laser desorption/ionization time of flight analysis (MALDI-TOF).

The invention also comprises compositions. In one embodiment, the invention comprises a dendrimer to which is linked one or more abortive promoter cassettes. In one embodiment, the multiple abortive promoter cassettes linked to said dendrimer are identical. In another, the multiple abortive promoter cassettes linked to said dendrimer are nonidentical.

In further embodiments, the invention comprises a dendrimer to which is linked one or more of abortive promoter cassettes, which is further linked a second molecule with specificity for a target molecule of interest. Such second molecules may be selected from the group consisting of: a DNA sequence; a RNA sequence; a PNA sequence; an antibody; a binding protein; a signalling molecule; a hapten; biotin; a binding nucleoside; a binding nucleotide; an enzyme substrate; an enzyme substrate derivative; and an ion.

In some embodiments, the invention comprises a kit containing a dendrimer to which is linked one or more of abortive promoter cassettes. In another embodiment, the invention is a composition or device for performing the methods of the invention.

In some embodiments, the invention comprises a method for detecting the presence of a target molecule comprising: synthesizing multiple copies of oligonucleotides through abortive reiterative synthesis on a nucleic acid template; introducing said multiple copies of abortive reiteratively synthesized transcripts into a signal amplification cascade; and determining the presence of the signal from said signal cascade, thereby detecting the presence of said target molecule. The signal cascade includes, but is not limited to: PCR; Abortive transcription; FRET; Horseradish peroxidase; Alkaline Phosphatase; and other Enzymatic amplification of the signal triggered by the synthesized transcripts.

In another embodiment, the invention comprises a method for detecting the presence of one or more target molecules comprising synthesizing multiple copies of oligonucleotides through abortive reiterative synthesis on a nucleic acid template; simultaneously performing one or more different syntheses of multiple copies of oligonucleotides through abortive reiterative synthesis on a nucleic acid template; determining the presence of said of one or more target molecules.

### BRIEF DESCRIPTION OF THE DRAWINGS

FIG. 1: Abortive Promoter Cassettes. Abortive Promoter Cassettes (APC) are regions of nucleic acid that form a polymerase binding site and can be attached to other macromolecules through interaction with a specific nucleic acid sequence, which is termed the APC linker. APC linkers can be attached to target nucleic acids (DNA or RNA) by hybridization to complementary sequences on either the template or non-template strands of the target nucleic acid. An APC Linker can also hybridize to a complementary sequence placed on any target molecule, such as a protein, for detection of molecules that bind to said protein. Multiple detectable oligonucleotides are generated by polymerase bound to the Abortive Promoter Cassette. In this figure, the APC depicted contains two regions of essential complementary (A, A′ and C, C′), which are separated by a “bubble region.” In this schematic, the “bubble region” is generated because regions of the two strands are non-complementary (B, and E). Alternatively, the APC may have two completely complementary strands. Upon binding of the RNA polymerase, the DNA strands separate, which leads to the formation of the “bubble region.”

Regions A, B, and C are on one strand. Regions C′, E, and A′ are on the complementary strand. The APC may be made from two separate strands (ABC and C′EA′) or all 6 regions may be on a single polynucleotide, in which regions C and C′ are separated by a linker region D, which can modified to be as long as needed. Linker region D may serve only to join C and C′ or the sequence of region D may serve as a binding site for other factors that may enhance abortive transcription, such as transcription roadblock proteins, including but not limited to EcoRI QIII mutant, the lac repressor and other RNA polymerases. The linker region D may be designed for a single road block protein, or multiple roadblock proteins. The length of linker region D will depend on the function of the linker region.

FIG. 2: Signal Generation by Reiterative oligonucleotide synthesis. A signal is generated by the enzymatic incorporation of nucleotides to form multiple oligonucleotide transcripts. Under appropriate conditions, RNA oligonucleotides can be made from nucleic acid templates in the absence of a promoter. An initiator may be comprised of one or more nucleosides, nucleoside analogs, nucleotides, or nucleotide analogs. The initiator may contain one or more covalently joined nucleotides, including but not limited to, 1-25 nucleotides, 26-50 nucleotides, 51-75 nucleotides, 76-100 nucleotides, 101-125 nucleotides, and 126-150 nucleotides, 151-175 nucleotides, 176-200 nucleotides, 201-225 nucleotides, 226-250 nucleotides and more than 250 nucleotides, and may contain a functional R group. The initiator (n copies) can be elongated directly with n copies of a terminator to end chain elongation or n copies of other elongator nucleotides (Y positions) may be incorporated between the initiator and the terminator to form a longer oligonucleotide. The terminator may contain a second functional group. N₁=Initiating mononucleotide or oligonucleotide analog, N_{E}=Elongating mononucleotides or analog, N_{T}=Terminating mononucleotide or analog, Z=x+y; R₁=H, OH, or reporter group; R₂=H, OH, or reporter group; N=deoxy or ribonucleotides; Polymerase=RNA-dependent or DNA-dependent RNA polymerase. DNA or RNA may be attached to other molecules, such as proteins.

The reiterative oligonucleotide transcripts are then detected using mass spectrometry. Mass spectrometry may be performed on oligonucleotide transcripts that do not comprise any “labeling” moeity. However, the use of nucleotide analogs in any one of the initiator, incorporated nucleotides or terminator molecules will change the molecular weight of the resultant species and may be used in mass spectrometry to differentiate multiple oligonucleotides of the same nucleotide length.

FIG. 3: Dinucleotide synthesis via abortive initiation on single-stranded DNA or RNA. Single stranded (ss) nucleic acid is DNA or RNA. Polymerase is a DNA-dependent or RNA-dependent RNA polymerase. N₁=3′-OH nucleoside or nucleotide initiator; N_{T}=5′-triphosphate nucleotide or nucleotide analog terminator. R₁ may be on the 5′ phosphate group, the 2′ position of the sugar, or on the purine or pyrimidine base. R₂ may be on the pyrimidine or purine base or 2′ or 3′ position of the sugar/ribose or deoxyribose. R₁=H, OH, and/or any reporter group or reporter group precursor, as described herein. R₂=H, OH, and/or any reporter group or reporter group precursor, as described herein.

FIG. 4: Target Site Probe. An RNA polymerase can be directed to specific nucleotide positions (sites) in target nucleic acids by the hybridization of a gene-specific or region-specific Target Site Probe (TSP). The target site is a nucleotide position in the DNA to be analyzed for sequence (as in detection of single nucleotide polymorphisms) or structure (as in assessing the methylation status of a specific nucleotide), and it is located on the template strand of the target sequence at the junction of regions E and C′ in the target sequence. The TSP contains a region of homology to the target nucleic acid (Region A) which begins approximately 8-14 nucleotides and ends approximately 15-35 nucleotides upstream of the target site nucleotide. A second region of the TSP is designed to be non-complementary to the 8-14 nucleotides immediately upstream of the target site (Region B), so that a melted “bubble” region forms when the TSP hybridizes to the target nucleic acid. The TSP contains a third region (Region C) which is essentially complementary to the 5-25 nucleotides immediately downstream of the target site nucleotide. RNA polymerase will bind to the bubble complex such that transcription will start at the E/C′ junction and will move downstream into the C/C′ hybrid.

FIG. 5: Deamination conversion of unmethylated cytosine groups in DNA. Deamination converts unmethylated C to U. Methylated C groups, such as those in CpG islands that regulate eukaryotic genes, are resistant to deamination and remain as C in the product DNA. If 100% deamination occurs, methylated DNA will still contain CpG doublets, whereas unmethylated DNA will contain no cytosine and will now contain UpG where CpG doublets were before deamination. This difference in DNA sequence can be used to distinguish between methylated and unmethylated DNA by abortive transcription because the two DNAs encode different dinucleotides.

Dinucleotide synthesis can be used to assess the overall methylation state of DNA. In the presence of RNA polymerase, CTP or a CTP analog (R₁-C—OH), and GTP or a GTP analog (R₁-CpG-R₂), the deaminated methylated DNA template will produce n copies of a labeled dinucleotide product, where n is proportional to the number of methylated CpG dinucleotides in the starting DNA. The deaminated unmethylated DNA template can produce no dinucleotide with these substrates because the template no longer encodes “C” at any position.

Abortive synthesis of trinucleotides by transcription initiation with labeled dinucleotides that end in C (ApC, CpC, GpC, UpC) and termination with GTP can be used to produce signal from the deaminated methylated template, but not the deaminated unmethylated template. This trinucleotide synthesis approach may be expanded by the addition of a site-specific oligonucleotide to allow assessment of the methylation status of a specific CpG site, rather than the entire island, as illustrated in FIG. 6.

FIG. 6: Assessing methylation status of specific CpG sites in CpG islands by abortive initiation. Target site probes can be used to examine the methylation status of specific CpG islands in specific genes. In the deaminated methylated DNA, the dinucleotide CpG is encoded by the template at the 3 methylated sites 1, 3 and 4, but not by the unmethylated site 2. To specifically determine if Site 3 is methylated and if so, to what extent, position (C21) can be targeted with a Target Site Probe, as described in FIG. 4. The template C in question is positioned at the junction of the bubble region and the downstream duplex so that it encodes the next incorporated nucleotide for appropriately primed RNA polymerase that binds to the bubble region. If initiator R₁-NₓpC—OH is used, where Nₓ may be C for a dinucleotide CpC initiator or Nx may be CpC for a trinucleotide initiator, etc., the initiator can be elongated with GTP or a GTP analog pppG-R2G to form a trinucleotide R₁NₓCpG-R₂. Similarly, if the C in question was not methylated, the position will now be a U and will encode nucleotide A. If ATP or an ATP analog pppA-R_{2A} is present, it will be incorporated opposite positions where the C was not methylated.

Mass spectrometry, which is able to readily distinguish different trinucleotide sequences on the basis of the molecular mass, is performed to determine the relative amount of oligonucleotides terminating at G compared with those terminating at A. The ratio of the magnitude of the peak corresponding to the G-terminating trinucleotide to the total peak of both the A-terminating and G-terminating trinucleotides corresponds to the methylation index (M). If all of the Cs at that position are methylated, M=1. If none of the site is methylated, M=0. Again, nucleotide analogs may also be used in the invention to impart different molecular weights to the resultant oligonucleotides.

FIG. 7: Detection and identification of single nucleotide polymorphisms (SNPs) by abortive transcription. The identity of a specific DNA nucleotide (A,C,G,T/dU) can be identified by abortive transcription with the use of a Target Site Probe (TSP). For example, to determine whether a DNA contains a normal nucleotide (wild type) or a mutant nucleotide (point-mutation, single nucleotide polymorphism/SNP), a gene-specific TSP can be added to target DNA (or amplification/replication product) such that the SNP position corresponds to the last nucleotide in the C/C′ hybrid at the junction of the downstream duplex and the bubble region. An initiator oligonucleotide (R₁NI—OH) that is complementary to the region upstream of the SNP site can be elongated by an RNA polymerase to add the next encoded nucleotide, corresponding to the SNP. The resultant oligonucleotides may be detected, and distinguished from each other, with mass spectrometry.

FIG. 8: Indirect detection of nucleic acids, proteins and the like by abortive transcription. Abortive transcription may be used to directly detect nucleic acids, such as DNA or RNA associated with specific diseases or with viral and bacterial pathogens, by synthesis of reiterative oligonucleotide transcripts. Alternatively, one may detect the presence of a nucleic acid, protein, carbohydrate, lipid, hapten or other molecule indirectly by linking a) an antibody, complementary nucleic acid sequence, chemical moeity and the like that recognizes said protein, carbohydrate, lipid, hapten or other molecule to b) one or more Abortive Promoter Cassettes (APC). The production of multiple reiterative oligonucleotide transcripts from the APC may be detected, and indicates the presence of the target molecule.

The APC may contain an artificial promoter, or it may contain the promoter for a specific RNA polymerase. Enhanced detection of molecular targets may be achieved by the use of particles to which multiple copies, including tens, hundreds, thousands, tens of thousands or even more of the Abortive Promoter Cassettes (APC) have been attached. FIG. 8 illustrates the use of an APC coupled a capture sequence for detection of a nucleic acid.

FIG. 9. RNA or DNA targets can be detected by hybridizing chemically-modified sequence-specific target site probes. While a nucleic acid target may be detected by abortive transcription directly on the target molecule, it may also be detected by hybridization to a probe sequence to which is attached an Abortive Promoter Cassette (APC). Multiple APCs may be attached, increasing the signal generation from a single binding event, and therefore the sensitivity. Sensitivity and specificity may also be increased by multiplexing: generating multiple different signals from one reaction. Three probes, each hybridizing to different regions of the same target molecule, and each labelled with different APCs, are hybridized to a target nucleic acid. Sensitivity is increased by the use of three APC over one. Specificity is also increased, as the production of three independent signals from one target molecule reduces false positives that may occur.

Dendrimers containing multiple APCs are attached to the target-site probe via a heterobifunctional linker. Each dendrimer contains multiple copies of the same APC and encodes a single abscript. Multiple dendrimers can be synthesized, each containing a different APC. All APCs can be detected and quantified simultaneously. Specificity will be determined by looking at the ratio of each of the different APC products. Increased specificity can be obtained by using additional dendrimers tagged with APCs that encode different abscripts. Increased sensitivity can be achieved by using multiple dendrimers, each encoding the same product.

It is also possible to attach APC-tagged dendrimers to a protein, such as an antibody.

FIG. 10. As with nucleic acids, multiplexing may improve sensitivity and specificity. The use of a capture antibody may further improve specificity (as only specific proteins will be bound) and may also improve sensitivity, as the capture antibody may extract the target molecule from a large volume of sample. In the illustrated embodiment, protein toxins are immobilized with a capture antibody. Two more toxin epitope-specific peptide antibodies, each tagged with an abortive promoter cassette, are added. Abscription substrates are added such that each APC makes a different abscription product, which are then detected and quantified. A higher level of specificity may be achieved by adding more toxin-specific antibodies. A positive signal would then require abscript production from each of these antibodies in the appropriate ratio. For example, APC1 encodes ApUpA, APC2 encodes ApUpC, APC3 encodes ApUpG. All three abscripts use the same ApU initiator. Each incorporates a different nucleotide as position three, thereby producing abscripts of different mass. Alternatively, A, C and G may be differently labelled, each producing a different signal.

FIG. 11. Detection of telomerase activity with reiterative oligonucleotide synthesis. Reiterative oligonucleotide synthesis with DNA polymerases can also be used for signal generation, however, the product oligonucleotides need not be released, but may be joined tandemly in the product. As an example, telomerase activity can be detected by immobilizing a telomerase-specific probe to a solid matrix to capture cellular telomerase, which carries its own RNA template for DNA synthesis. For example, with human telomerase, the RNA template on the enzyme encodes the DNA sequence GGGTTA. The capture probe may contain the sequence GGGTTA, which will be added reiteratively to the end of the telomerase capture probe, if telomerase is present in the sample. Signal generation can be achieved in several ways, one of which involves including one or more reporter tagged dNTPs in the synthesis reaction to produce a product that has multiple R₁ groups attached along the backbone of the DNA product. FIG. 11 shows template sequences for the abortive transcription reactions. FIG. 11a: Poly[dG-dC] is a synthetic deoxyribonucleotide polymer of repeating dCpdG. Individual strands contain variable numbers of dinucleotide repeats. FIG. 11b: Bubble complex 1 was made by annealing synthetic, partially complementary template and non-template strands. The vertical offset of the non-template strand represents the single-stranded, bubble portion of the molecule. The coordinate system is based on the downstream edge of the bubble. The unpaired bases next to the double-stranded segment are at position +1. Positions to the left (upstream) of position +1 are given negative numbers starting with −1. The coordinate system is used to indicate the position of the 3′ ends of the ribonucleotide initiators. The 3′ end of initiator AA is aligned at +1 and the 3′ end of initiator AU is aligned at +2. The transcription reaction proceeds from left to right from 3′ end of the initiator, according to theory. FIG. 11c represents the template strand without the complementary non-template strand. The sequence is shown in the 3′ to 5′ orientation.

FIG. 12: Some nucleotides that can be elongators or terminators, for use in the methods and compositions of the invention. Nucleotide analogs that may be included at internal or 3′ terminal positions in oligonucleotides are shown. All of these analogs can be converted to terminators simply by replacement of the 3′ OH group. These analogs may also function as terminators with the 3′ OH group under particular reaction conditions.

FIG. 13: Other fluorescent groups that may be R. Such groups would be useful in the methods and compositions of the invention after addition onto the 5-S- position in a pyrimidine or the 8-S- position in a purine.

FIG. 14: Other R group modifications of nucleotides: 5-S-substituted pyrimidines or 8-S-substituted purines of the formula NucSR, wherein a is 1 to 2.

FIG. 15: Multiplex DNA detection. Multiple target specific probes, each hybridizing to a different region of a target DNA molecule, and as close as approximately 100 nucleotides, are added to a sample containing the target DNA molecule. If the TSPs encode abortive transcription products with the same sequence and reporter, this method will result in increased sensitivity.

If the TSPs encode abortive transcription products with different sequences and reporters, high selectivity and multiplexing may occur.

FIG. 16: Modular APCs. An APC may be designed with a TSP linker, or with a nucleic acid probe that is specific for the target sequence. If the APCs encode abortive transcription products with the same sequence and reporter, this method will result in increased sensitivity.

If the APCs encode abortive transcription products with different sequences and reporters, high selectivity and multiplexing may occur.

FIG. 17: Protein detection using APCs.

FIG. 18. Comparison of mass spectra from 50 pmols of AUG and AAG trinucleotides.

FIG. 19. Sensitivity of Mass Spectrometry method for detecting AAG trinucleotide at 50, 5, and 0.5 pmol in 50% CH₃CN/H₂O.

FIG. 20(A) Mass spectra summed over scans 190-210 (˜3.5-3.8 min.) for abscription reactions spiked with 10 and 100 μM AAG. The AAG signal near 468 Da arises from the −2 charged ion.

FIG. 20(B) Chromatograms for the signals from mass range 468-469 Da (AAG) and 593-594 Da (ApA) from the 100 μM spiked sample.

FIG. 21. Abortive promoter cassettes used in example multiplexing reaction. The bubble part of the abscription complex is shown in bold and the initiation site on the template strand is highlighted in gray. Both abortive promoter cassettes were constructed with the same target site probe (TSP), A192, but with different template strands (TEM), A197 or A56. The initiator for bubble 4 is ApA and for UC2361 it is UpC and the incorporating nucleotide is GTP. The expected abscript product from the bubble 4 is AAG and from UC2361 is UCG.

FIG. 22. Analysis of abscription products from the mixed APC experiment by TLC. Lanes 1-3 have bubble 4 (100 fmol final) and were initiated with ApA (lane 1), UpC (lane 2), ApA+UpC (lane 3). Lanes 4-6 have equimolar concentration of bubble 4(100 fmol final) and APC UC2361(100 fmol final) and they were initiated with ApA in lane 4, with UpC in lane 5 and with both ApA+UpC in lane 6. Lanes 7-9 have APC UC2361(100 fmol final) and initiated with UpC (lane 7), ApA (lane 8) and ApA+UpC (lane 9). Lane 10 is the TEM A197 initiated with ApA, lane 11 is the TEM A57 initiated with UpC and lane 12 is the control for no APC with initiators ApA+UpC. Lane 13 is the control for the α³²P-GTP. The initiator concentrations were 1 mM final for each.

FIG. 23. Representation of 10 Abortive Promoter Cassettes made with different combinations of oligonucleotide strands, for use in multiplex abscription reactions. The bubble part of the abscription complex is shown in bold and the initiation site on the template strand is highlighted in gray. The identity of each target site probe (TSP), and template strand (TEM) used to construct the APC is listed alongside the APC. Also listed is the initiator and expected abscription product.

FIG. 24. The annealing of oligonucleotide strands to form the APCs depicted in FIG. 23 was determined by electrophoresis on a 4% agarose gel. 4% Agarose gel analysis of the APCs. 5 μl of 1 pmol/μl of the APC was loaded into each lane (1-10). To show the comparison with the single strand, TEM A191 (5 μl of 50 pmol/μl) was loaded in lane 11 and lane 12 has the 50 bp ladder.

FIG. 25. TLC analysis of the abscripts made from the APCs 4, 12, 24, 25 and 32. Each abscription was done with 100 fmol of APC with 1 mM final concentration of the initiator along with 1 mM final concentration of the NTP as listed in the figure along with 1 μl of the abscriptase. The abscription was done for 60 minutes at 45° C. 2 μl from each reaction was spot on the TLC plate and developed with 6:3:1 of Isopropanol:NH₄OH:H₂O. The TLC plate was exposed to phosphor imager screen for 10 minutes.

FIG. 26. Concentration of SEB protein in sandwich ELISA (x-axis) against abscript generated (y-axis).

FIG. 27. Combined chromatogram of 10 trinucleotides from their individual runs. The embedded chart shows which numbered peak refers to which abscript, as well as the relevant retention time, m/z value, and peak area. 10 ul of a 10 uM solution was analyzed (100 pmol) by Atlantis dC18 3um 2.1 mm X30 mm column. The MS tune file used was “abscript”.

FIG. 28. Mixture of abscripts detected simultaneously by mass spectroscopy. Each abscript is shown next to its corresponding m/z ratio. 10 ul of a 10 uM solution was analyzed (100 pmol) by Atlantis dC28 3 um 2.1 mm X 30 mm column. The MS tune file used was “abscript”.

FIG. 29. Representation of two Abortive Promoter Cassettes used in multiplex abscription reactions. The bubble part of the abscription complex is shown in bold and the initiation site on the template strand is highlighted in gray. The identity of each target site probe (TSP), and template strand (TEM) used to construct the APC is listed alongside the APC. Also listed is the initiator and expected abscription product.

FIG. 30. Total signal generated from multiplex abscription reaction compared with control reactions.

FIG. 31. Mass spectrogram from the multiplex reaction with two APCs; APC 2-1 and APC 12-1. The peak at 450.0895 corresponds to AAU and the peak at 469.5732 corresponds to AAG. An Atlantis dC18 3 um 2.1 mm X30 mm column, was used, and the MS tune file used was “abscript”.

### DETAILED DESCRIPTION OF THE INVENTION

The invention provides methods for detecting the presence of a target molecule (such as nucleic acid sequence or protein) through reiterative synthesis on a nucleic acid template by generating multiple oligonucleotides, which are detected with mass spectrometry. The methods generally comprise using a nucleotide or oligonucleotide transcription initiator to initiate synthesis of an abortive oligonucleotide product that is substantially complementary to a site on a target nucleic acid; terminating the polymerization reaction (e.g. by nucleotide deprivation, incorporation of a chain terminating nucleotide, or through the presence of a transcription termination signal in the template sequence); and, optionally, using either (1) a target site probe to form a transcription bubble complex which comprises double-stranded segments on either side of a single-stranded target site or (2) an abortive promoter cassette comprising a transcription bubble region which includes a target site or (3) an abortive promoter cassette that is attached to any target molecule and then used to generate a signal. The signal comprises multiple oligonucleotide transcripts that are detected by mass spectrometry.

In accordance with one aspect, the invention provides methods of detecting a target molecule using multiple abortive oligonucleotide transcripts from portions of a target DNA or RNA sequence, wherein the methods comprise combining and reacting the following: (a) a single-stranded target nucleic acid comprising at least one target site; (b) an RNA initiator that is complementary to a site on the target nucleic acid that is upstream of the target site; (c) an RNA polymerase; (d) optionally, nucleotides and/or nucleotide analogs; (e) a chain terminator; and (f) optionally, either (1) a target site probe that partially hybridizes to a target region on the target nucleic acid, forming a transcription bubble complex that includes first and second double-stranded regions on either side of a single-stranded target site or (2) an abortive promoter cassette comprising a transcription bubble region that includes a transcription start site. The combination is subjected to suitable conditions, as described below, such that (a) a target site probe hybridizes with a target nucleic acid in a target region that includes the target site; (b) an RNA initiator hybridizes upstream of a target site; (c) an RNA polymerase utilizes the RNA initiator to initiate transcription at the target site, elongation occurs, and an oligonucleotide transcript is synthesized; (d) a chain terminator terminates transcription during elongation; (e) the RNA polymerase releases the short, abortive oligonucleotide transcript without substantially translocating from the polymerase binding site or dissociating from the template; and (f) (c)-(e) are repeated until sufficient signal is generated and the reaction is stopped. Alternatively, (a) an abortive promoter cassette hybridizes with an end of the target nucleic acid; (b) an RNA initiator hybridizes upstream of a transcription start site; (c) an RNA polymerase utilizes the RNA initiator to initiate transcription at the target site, elongation occurs, and an oligonucleotide transcript is synthesized; (d) a chain terminator terminates transcription during elongation; (e) the RNA polymerase releases the short, abortive oligonucleotide transcript without substantially translocating from the polymerase binding site or dissociating from the template; and (f) (c)-(e) are repeated until sufficient signal is generated and the reaction is stopped.

The multiple oligonucleotide transcripts thus generated, i.e. the signal, are detected and quantified by mass spectrometry, indicating the presence of the target molecule. In some aspects, the methods of the invention may also indicate the nature of the target molecule, or the nature of a pool of target molecules.

General Techniques

The practice of the present invention will employ, unless otherwise indicated, conventional techniques of molecular biology (including recombinant techniques), microbiology, cell biology, biochemistry, and immunology, all of which are well within the skill of the ordinary practioner in the art.

Terms

To facilitate understanding of the invention, the following terms have the following meanings unless expressly stated otherwise. Additional elaboration of many of the terms used herein may be found in International Application WO 03/038042, published May 8, 2003 and U.S. Application Publication Number US-2003-0099950, published May 29, 2003; and pending U.S. Application Ser. No. 10/425,037, filed Apr. 29, 2003.

“About” as used herein means that a number referred to as “about” comprises the recited number plus or minus 1-10% of that recited number. For example, “about” 50 nucleotides can mean 45-55 nucleotides or as few as 49-51 nucleotides depending on the situation.

“Abortive transcription” is an enzyme-mediated process that reiteratively initiates and terminates the synthesis of oligonucleotides that correspond to at least one portion, or target site, of a complementary nucleic acid template sequence. The abortive oligonucleotides synthesized vary in length of nucleotides, and may contain from about 2 to about 26 nucleotides, about 26 to about 50 nucleotides and about 50 nucleotides to about 100 nucleotides, and greater than 100 nucleotides.

“Abortive transcription” may include three phases, namely, initiation, elongation, and termination. During the initiation phase, a polymerase forms a phosphodiester bond between an initiator and a second NTP, and then adds subsequent NTPs, et cetera, transcribing the template sequence to synthesize an oligonucleotide transcript of from about 2 to about 50 nucleotides in length and then terminating the transcription event by releasing the nascent oligonucleotide transcript, without the polymerase substantially translocating from the polymerase binding site or dissociating from the template. In other words, the RNA polymerase substantially remains at the initial binding site on the template, releases a first nascent oligonucleotide transcript, and then is capable of engaging in another transcription initiation event to produce a second oligonucleotide transcript, which is substantially complementary to substantially the same target site that was transcribed to produce the first oligonucleotide transcript. In this manner, the polymerase reiteratively transcribes a single portion of the template (i.e., a target region) and releases multiple copies of substantially identical nascent oligonucleotide transcripts.

“Reiterative” refers to multiple identical or highly similar copies of a sequence of interest.

“Oligonucleotide product” refers to the oligonucleotide that is synthesized by the reiterative synthesis reaction of the present invention. An oligonucleotide product may be an “oligonucleotide transcript,” if the polymerization reaction is a transcription reaction catalyzed by an RNA polymerase, or an “oligonucleotide repeat,” if the polymerization reaction is a DNA synthesis reaction catalyzed by telomerase or DNA polymerase. The term “oligonucleotide” generally refers to short, typically single-stranded, synthetic polynucleotides that are comprised of two or more nucleotides and are generally, but not necessarily, less than about 200 nucleotides in length.

“Termination” refers to the use of a chain terminator to conclude a chain elongation or primer extension reaction that is catalyzed by a polymerase. A “chain terminator” or “terminator” may comprise any compound, composition, complex, reactant, reaction condition, or process (including withholding a compound, reactant, or reaction condition) which inhibits the continuation of transcription by the polymerase beyond the initiation and/or elongation phases. A “chain terminating nucleotide” is a chain terminator that comprises a nucleotide or nucleotide analog that inhibits further chain elongation once incorporated, due to either the structure of the nucleotide analog or the sequence of the nucleic acid being copied or transcribed.

A “target sequence” or “target polynucleotide” is a polynucleotide sequence of interest for which detection, characterization or quantification is desired. The actual nucleotide sequence of the target sequence may be known or not known. The “target sequence” may or may not be the actual molecule of interest.

A “target site” is that portion of the target sequence that is detected by transcription by a polymerase to form an oligonucleotide product. In accordance with the invention, there is at least one target site on a target nucleic acid. The sequence of a target site may or may not be known with particularity. That is, while the actual genetic sequence of the target nucleic acid may be known, the genetic sequence of a particular target site that is transcribed or replicated by a polymerase need not be known.

A “target region” is that portion of a target sequence to which a target site probe partially hybridizes to form a bubble complex, as described in detail below. In accordance with the invention, there is at least one target region on a target nucleic acid, and each target region comprises a target site. The sequence of a target region is known with sufficient particularity to permit sufficiently stringent hybridization of a complementary target site probe, such that the target site probe forms a bubble complex with the target region.

Generally, a “template” is a polynucleotide that contains the target nucleotide sequence. In some instances, the terms “target sequence”, “template polynucleotide”, “target nucleic acid”, “target polynucleotide”, “nucleic acid template”, “template sequence”, and variations thereof, are used interchangeably. Specifically, the term “template” or “template strand” refers to a strand of nucleic acid on which a complementary copy is synthesized from nucleotides or nucleotide analogs through the activity of a template-dependent nucleic acid polymerase.

“Nucleotide” includes cytosine (C), thymine (T), and uracil (U), adenine (A) and guanine (G) nucleotides and analogs.

The term “initiator” refers to a mononucleoside, mononucleotide, oligonucleotide, polynucleotide or analog thereof, which is incorporated into the 5′ end of a nascent RNA molecule and may be considered a “primer” for RNA synthesis (“initiator primer”).

“Incorporation” refers to becoming a part of a nucleic acid polymer. There is a known flexibility in the terminology regarding incorporation of nucleic acid precursors. For example, the nucleotide dGTP is a deoxyribonucleoside triphosphate. Upon incorporation into DNA, dGTP becomes dGMP, that is, a deoxyguanosine monophosphate moiety. Although DNA does not include dGTP molecules, one may say that one incorporates dGTP into DNA.

“Identity” or “identical” is used herein as commonly understood in the art. For example, a polynucleotide having a nucleotide sequence at least, for example, 95% “identical” to a reference nucleotide sequence means that the nucleotide sequence of the polynucleotide is identical to the reference sequence over the region of interest except that the polynucleotide sequence may include up to five mismatches per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The reference (query) sequence may be the entire nucleotide sequence shown or any polynucleotide fragment thereof.

As a practical matter, whether any particular nucleic acid molecule is at least 80%, 85%, 90%, 92%, 95%, 96%, 97%, 98% or 99% identical to, for instance, the nucleotide sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The identity between a reference (query) sequence (a sequence of the present invention) and a subject sequence, also referred to as a global sequence alignment, may also be determined using the FASTDB computer program based on the algorithm of Brutlag et al. (Comp. App. Biosci. 6:237-245 (1990)). Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter. According to this embodiment, if the subject sequence is shorter than the query sequence because of 5′ or 3′ deletions, not because of internal deletions, a manual correction is made to the results to take into consideration the fact that the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. A determination of whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of this embodiment. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are made for the purposes of this embodiment.

The term “label” refers to any atom, molecule, or moiety which can be used to provide a detectable signal, either directly or indirectly, and which can be attached to a nucleotide, nucleotide analog, nucleoside mono-, di-, or triphosphate, nucleoside mono-, di-, or triphosphate analog, polynucleotide, or oligonucleotide. In some embodiments the label is detected by the alteration in mass that the labeling moeity imparts to the resultant oligonucleotide. The detectable molecule may also be quantifiable. Such mass-labels may also be detected by other means known in the art.

The term “multiplex” means producing or relating to a system of producing several messages or signals simultaneously from the same reaction.

The term “Mass Spectrometry” refers to the analytical method for determining the relative masses and/or relative abundances of components in a beam of ionized molecules or molecular fragments produced from a sample in a high vacuum. The term as used herein includes, inter alia, fast atomic bombardment (FAB) mass spectrometry, plasma desorption (PD) mass spectrometry, electrospray/ionspray (ES) mass spectrometry, matrix-assisted laser desorption/ionization (MALDI) mass spectrometry, matrix-assisted laser desorption/ionization time of flight analysis (MALDI-TOF), and may be combined with other technologies, including electrophoresis, liquid chromatography, high speed gel filtration, etc. Mass spectrometric detection may be augmented by incorporating into the nucleic acids isotopes, molecules, or atoms which normally do not occur in DNA such as S, Br, I or Ag, Au, Pt, Os, Hg.

The term “dendrimer” (from the Greek dendra for tree) refers to a branched structure which is, typically, a manufactured or synthesized branched polymer.

Components and Reaction Conditions

Target Nucleic Acid

The target nucleic acid can be either a naturally occurring or synthetic polynucleotide segment, and it can be obtained or synthesized by techniques that are well-known in the art. A target sequence to be detected in a test sample may be present initially as a discrete molecule, may be present as only one component of a larger molecule, a minor or major fraction of a complex mixture, such as a biological sample. The target nucleic acid to be detected may include nucleic acids from any source, in purified, or unpurified form, which can be DNA (including double-stranded (ds) DNA and single-stranded (ss) DNA) or RNA (including tRNA, mRNA, rRNA), mitochondrial DNA or RNA, chloroplast DNA or RNA, DNA-RNA hybrids, or mixtures thereof, genes, chromosomes, or plasmids; and DNA from the genomes of biological material, such as the genomes of microorganisms, plants, animals, humans. Standard techniques in the art may be used to obtain and purify the nucleic acids from a test sample. Detection of an RNA target may or may not require initial complementary DNA (cDNA) synthesis, as known in the art. Detection of a DNA-RNA hybrid may require denaturation of the hybrid to obtain a ssDNA or denaturation followed by reverse transcription to obtain a cDNA.

Other Target Molecules

In another embodiment of the invention, the target may be another molecule, such as a protein, which is labeled by covalent or noncovalent attachment of a nucleic acid sequence which can be used for reiterative oligonucleotide synthesis. For example, a molecule may be detected by an antibody coupled to a nucleic acid sequence which may serve as the template for synthesis of multiple reiterative oligonucleotides. The target molecule to be detected may include proteins, peptides, carbohydrates, lipids, haptens or other molecules from any source, in purified or unpurified form. Standard techniques in the art are used to obtain and purify the molecules from a test sample.

Immobilization

In one embodiment of the invention, the target molecule may be immobilized on a substrate. In another embodiment, the target molecule may be immobilized to form, for example, a microarray. A single molecule array in accordance with this embodiment includes a solid matrix, a bioreactive or bioadhesive layer, and a bioresistant layer. Solid phases that are useful as a matrix for the present invention include, but are not limited to, polystyrene, polyethylene, polypropylene, polycarbonate, or any solid material in the shape of test tubes, beads microparticles, dip-sticks, membranes, microtiter plates, test tubes, and Eppendorf tubes, glass beads, glass test tubes, and any other appropriate shape that is made of glass. In general, suitable solid matrices comprise any surface to which a bioadhesive layer, such as a ligand-binding agent, can be attached or any surface which itself provides a ligand attachment site.

Antibody or oligonucleotide capture probes can be attached to a bioadhesive pattern by methods known in the art including providing a polyhistidine tag or crosslinking reagents.

In one embodiment, a solid matrix may be housed in a flow chamber having an inlet and outlet to accommodate the multiple solutions and reactants that are allowed to flow past the immobilized capture probes. The flow chamber can be made of plastic or glass and may be either open or transparent in the plane viewed by a microscope or optical reader. Electro-osmotic flow includes a fixed charge on the solid support and a voltage gradient (current) passing between two electrodes placed at opposing ends of the solid support.

Primers

In accordance with an embodiment of the invention, a primer is used to initiate transcription by a polymerase of a target site on the target nucleic acid and may be comprised of ribonucleotides or deoxyribonucleotides. In one embodiment, the primers are less than about 25 nucleotides in length, usually from about 1 to about 10 nucleotides in length, and preferably about 2 to 3 nucleotides in length. It may be desirable to modify the nucleotides or phosphodiester linkages in one or more positions of the primer.

Target Site Probes

In accordance with an embodiment of the invention, an oligonucleotide target site probe is used to direct a polymerase to a target site on the target nucleic acid (FIG. 4). A target site probe may also be referred to as the “nontemplate strand”. The target site probe may vary in the length of nucleotides, including but not limited to, about 20 to about 50 nucleotides, about 51 to about 75 nucleotides, about 76 to about 100 nucleotides, or greater than 100 nucleotides. A bubble complex comprises double-stranded regions on either side of a single-stranded region which includes a target site. A bubble complex in a target region may be formed by the structure of a target site probe. In one embodiment, the target site probe includes three regions: a first region on the 5′ end of the target site probe complementary to and hybridizing with the template sequence upstream of a target site on the template sequence; a second region, which is 3′ of the first region, is non-complementary to the template sequence and therefore does not hybridize with the template sequence; and a third region, which is on the 3′ end of the target site probe, is complementary to and hybridizes with the template sequence downstream of the target site. In another embodiment, the target site probe is substantially complementary to the single-stranded region, and the bubble complex is formed by the action of the polymerase.

Use of the target site probe directs the polymerase to a particular enzyme binding site (i.e., the double-stranded segment and bubble formed upstream of the target site by the template sequence and the primer) on the template sequence to facilitate the initiation of transcription at a particular target site. That is, rather than facilitating the random initiation of synthesis reactions by the polymerase along the length of a single-stranded template sequence, as described above, this embodiment provides targeted binding of the polymerase for the detection of a particular target site encompassed by the bubble complex formed by the target site probe.

The sequence of the target site probe will vary depending upon the target sequence. The overall length of the target site probe is selected to provide for hybridization of the first and third regions with the target sequence and optimization of the length of the second, non-hybridized region. The first and third regions of the target site probe are designed to hybridize to known sites on the target nucleic acid template. Depending upon the application, the sequence of the second region on the target site probe can be designed such that the second region may or may not be self-complementary.

In one embodiment, at least one target site probe is used to specifically initiate abortive oligonucleotide synthesis at one or more target sites on the nucleic acid template to produce multiple oligonucleotide products. In another embodiment, the target site probe directs the initiation of abortive transcription on a single-stranded target site in the absence of a template promoter sequence. See, for example, U.S. Pat. No. 5,571,669; Daube and von Hippel, Science, 258: 1320-1324 (1992).

Abortive Promoter Cassette (Also known as Abortive Bubble Cassette)

In accordance with the invention, an abortive promoter cassette (APC) may be used to link a target to a sequence to generate multiple detectable oligonucleotide products that indicate the presence of the target in a test sample. APCs may also be referred to as Abortive Bubble Cassettes (ABC). The APC is a self-complementary sequence of DNA that may consist of: (1) one contiguous oligonucleotide to which RNA polymerase can bind to form a transcription bubble; (2) two partially complementary upper and lower oligonucleotides that form a single-stranded transcription bubble region comprising a site from which an initiator and a suitable RNA polymerase can synthesize an abortive oligonucleotide product; or (3) two complementary oligonucleotides that form a transcription bubble region in the presence of an RNA polymerase, which allows for the synthesis of an abortive oligonucleotide product. The APC may contain an artificial promoter, or it may contain the promoter for a specific RNA polymerase. For example, trinucleotide or tetranucleotide products that could be generated with a common phage RNA polymerase can be made with a GpA or GpApA initiator and a pppG or pppA terminator.

In an exemplary embodiment, as illustrated in FIG. 4, the APC comprises eight regions, including an APC linker sequence which comprises either a 3′ or a 5′ single-stranded overhang region (i.e., a “sticky end”). A first region (A) on the 5′ end of the APC is complementary to a second region (A′) near the 3′ end of the APC. A third region (B) and a fourth region (E) are separated from each other by regions C, D, and C′ and are non-complementary to each other, such that the regions B and E form a single-stranded bubble region on the APC when the self-complementary regions of the APC interact with one another. Regions C and C′ are substantially self-complementary, such that the 5′ end of region C is complementary to the 3′ end of the region C′. Region D may be a short sequence joining C and C′ for a contiguous APC or may be a region comprising the free 3′ or 5′ ends of two separate upper and lower oligonucleotides for a two-part APC. Finally, the APC also includes an APC linker, a single-stranded region on either the 5′ end or the 3′ end of the APC oligonucleotide, which is formed through the complementary interaction of regions A and A′. The APC linker facilitates attachment of the APC with other target molecules, such as captured target DNA, RNA, or protein, for example.

A number of APC are embodied in the present invention. In some embodiments, the APC has the following features:
Upstream arm length>13 nt
Downstream arm length>13 nt
Bubble segment length=11 to 14 nt
Bubble sequence: Nontemplate strand consensus:

Upstream 5′ TANNNTN₅₋₈

Preferred sequence: 5′ TATAATN₅₋₈

Bubble sequence: Template strand Upstream 3° C(C or G or A)N₉₋₁₃ or A(C or G or A)N₉₋₁₃ which are preferred to T(C or G or A)N₉₋₁₃ or G(C or G of A)N₉₋₁₃.

In the following generic APC structures the underlined nucleotides are within the bubble, and “nn” represent initiation sites. The numbers shown below the first APC represent possible (alternative) initiator sites in the first APC:

1. The 2 most downstream sites within the bubble.

2. The site spanning the junction of the bubble and the downstream arm.

3. The downstream 2 nt immediately adjacent to the bubble.

4. The downstream site 1 nt offset from the edge of the bubble Similar initiator sites are found in the additional exemplary APCs, but are not shown.

```
5′ NNNNNNNNNNNNNTATAATNNNNNNNNNNNNNNNNNNNN
3′ NNNNNNNNNNNNNCCNNNNNNNNNNNNNNNNNNNNNNNN
                                                    nn 1.
                                                      nn 2.
                                                        nn 3.
                                                          nn 4.
5′ NNNNNNNNNNNNNTATAATNNNNNNNNNNNNNNNNNNNN
3′ NNNNNNNNNNNNNCGNNNNNNNNNNNNNNNNNNNNNNNN
5′ NNNNNNNNNNNNNTATAATNNNNNNNNNNNNNNNNNNNN
3′ NNNNNNNNNNNNNCANNNNNNNNNNNNNNNNNNNNNNNN
5′ NNNNNNNNNNNNNTATAATNNNNNNNNNNNNNNNNNNNN
3′ NNNNNNNNNNNNNACNNNNNNNNNNNNNNNNNNNNNNNN
5′ NNNNNNNNNNNNNTATAATNNNNNNNNNNNNNNNNNNNN
3′ NNNNNNNNNNNNNAGNNNNNNNNNNNNNNNNNNNNNNNN
5′ NNNNNNNNNNNNNTATAATNNNNNNNNNNNNNNNNNNNN
3′ NNNNNNNNNNNNNAANNNNNNNNNNNNNNNNNNNNNNNN
```

Specific non-limiting examples of APCs represented by the generic APC structures described above may be found in FIGS. 11, 21, 23 and 29. In some embodiments, the invention provides for APCs as described above, including in FIGS. 21, 23 and 29, but excluding APCs that comprise a target site probe with the sequence: 5′GGATACTTACAGCCATTATATTTAGCCC TACTCCATTCCATCCCGGGTTCGTCC-3′; and/or comprise a template strand with a sequence: 3° CCTATGAATGTCGGTACCTGTGCCGCTTATG AGGTAAGGTAGGGCCCAAGCAGG 5′; or the reverse or complimentary strands thereof.

The APC used in the practice of the invention may be made enzymatically or synthetically. The length of the APC is selected to optimize the stability of the bubble region and provide for the hybridization of the APC linker sequence with the target sequence, as has been described in WO 03/038042 and U.S. Application Publication Number US-2003-0099950, for example.

A single molecule with specificity for the target molecule of interest may be attached to one or multiple APCs. Typically, the same type of APC is attached to one molecule. In some embodiments, however, different APCs may be attached to the same target molecule. As illustrated in FIG. 9, a large number of APCs may be attached to a single molecule, greatly increasing the signal generated by a single binding event. APCs may be attached to a nucleic acid, antibody, or any other molecule.

In one embodiment, the invention provides for APCs that may be attached to any molecule desired by means of chemical coupling.

The invention also includes methods of detecting target molecules using the APCs of the invention.

Dendrimer Structures

Dendrimers are branched structures that, typically are artficially manufactured or synthesized branched polymers. For example, dendrimers may be made from acrylic acid and a diamine. Dendrimers may be obtained commercially from e.g. Dendritech, Inc. 3110 Schuette Drive, Midland, Mich. 48642.

Dendrimer synthesis is the field of polymer chemistry defined by regular, highly branched monomers leading to a monodisperse, tree-like or generational structure. Synthesizing monodisperse polymers demands a high level of synthetic control which is achieved through stepwise reactions, building the dendrimer up one monomer layer, or “generation,” at a time. Each dendrimer consists of a multifunctional core molecule with a dendritic wedge attached to each functional site. The core molecule is referred to as “generation 0.” Each successive repeat unit along all branches forms the next generation, “generation 1,” “generation 2,” and so on until the terminating generation.

There are two defined methods of dendrimer synthesis, divergent2 and convergent3 In the divergent method the molecule is assembled from the core to the periphery; while in the convergent method, the dendrimer is synthesized beginning from the outside and terminating at the core. In either method the synthesis requires a stepwise process, attaching one generation to the last, purifying, and then changing functional groups for the next stage of reaction. This functional group transformation is necessary to prevent unbridled polymerization. Such polymerization would lead to a highly branched molecule which is not monodisperse—otherwise known as a hyperbranched polymer. Although there are many uses for hyperbranched polymers, this essay does not investigate them.

In the divergent method, the surface groups initially are unreactive or protected species which are converted to reactive species for the next stage of the reaction. In the convergent approach the opposite holds, as the reactive species must be on the focal point of the dendritic wedge.

Due to steric effects, continuing to react dendrimer repeat units leads to a sphere shaped or globular molecule until steric overcrowding prevents complete reaction at a specific generation and destroys the molecule's monodispersity. The number of possible generations can be increased by using longer spacing units in the branches of the core molecule. The monodispersity and spherical steric expansion of dendrimers leads to a variety of interesting properties.

The steric limitation of dendritic wedge length leads to small molecular sizes, but the density of the globular shape leads to fairly high molecular weights. The spherical shape also provides an interesting study in molecular topology. Dendrimers have two major chemical environments, the surface chemistry due to the functional groups on the termination generation which is the surface of the dendritic sphere, and the sphere's interior which is largely shielded from exterior environments due to the spherical shape of the dendrimer structure. The existence of two distinct chemical environments in such a molecule implies many possibilities for dendrimer applications.

Theoretically, hydrophobic/hydrophilic and polar/nonpolar interactions can be varied in the two environments. The existence of voids in the dendrimer interior furthers the possibilities of these two heterogeneous environments playing an important role in dendrimer chemistry. Dendrimer research has confirmed the ability of dendrimers to accept guest molecules in the dendritic voids.

Dendrimers have found actual and potential use as molecular weight and size standards, gene transfection agents, as hosts for the transport of biologically important guests, and as anti-cancer agents, to name but a few. Much of the interest in dendrimers involves their use as catalytic agents, utilizing their high surface functionality and ease of recovery. Dendrimers' globular shape and molecular topology, however, make them highly useful to biological systems as well.

In some embodiments of the present invention, APCs are attached to branches of the dendrimer, typically by chemical coupling. The binding APCs on the surface of the dendrimer structure may result in a high density of APCs. One branch of the APC-dendrimer is linked to a second molecule, such as a probe, antibody, binding protein etc, with specificity for the target molecule of interest. As a result, binding of the second molecule to the target molecule of interest will result in a high density of APCs, and hence a large number of abortive oligonucleotide transcripts from the APCs. Thus, dendrimers enable large scale amplification of the signal associated with detection of a target molecule, with a resultant increase in sensitivity and (potentially) speed

Polymerase

Template-dependent polymerases for use in the methods and compositions of the present invention are known in the art and include both eukaryotic or prokaryotic polymerases, thermostable polymerase, DNA-dependent RNA-polymerase, DNA-dependent DNA-polymerase, RNA-dependent RNA-polymerase, RNA-dependent DNA-polymerase, DNA-dependent RNA-polymerase, the polymerase is able to tolerate label moieties on the phosphate group, the nuclease, and/or on the pentose ring of unincorporated nucleotides, polymerase capable of transcribing a single-stranded DNA template without a promoter sequence and other previously described.

In general, the enzymes included in the methods of the present invention preferably do not produce substantial degradation of the nucleic acid components produced by the methods.

Nucleotides

In accordance with the invention, the polymerase catalyzes a reaction in the usual 5′→3′ direction on the oligonucleotide product and either transcribes or replicates the target nucleic acid by extending the 3′ end of the initiator or primer through the sequential addition of nucleotides (NTPs), which may include nucleotide analogs (NTP analogs) and which may be labeled or unlabeled.

In some embodiments, the method uses a nucleotide that is derivatized with any one of an isotope, haptens, biotin, an enzyme (e.g horseradish peroxidase or alkaline phosphatase), a protein, an artificial promoter cassette, a photocrosslinker, a chemical crosslinker, steptavidin, a fluorescent moiety, a colorimetric moiety, a luminescent moiety, a chemiluminescent moiety, a metal (e.g. gold, silver), or a dye.

In other embodiments, the method uses a nucleoside, nucleotide, oligonucleotide or nucleic acid comprising a 5-S-substituted pyrimidine or 8-S-substituted purine of the formula NucSR;

wherein Nuc is pyrimidinyl or purinyl;

wherein S is sulfur;

wherein R is selected from the group consisting of H, haptens, biotin, an enzyme (e.g horseradish peroxidase or alkaline phosphatase), a protein, an artificial promoter cassette, a photocrosslinker, a chemical crosslinker, steptavidin, a fluorescent moiety, a colorimetric moiety, a luminescent moiety, a chemiluminescent moiety, a metal (e.g. gold, silver), a dye, a nucleic acid cellular uptake group, a C₆₋₁₀ aryl, C₆₋₁₀ ar(C₁₋₆)alkyl, C₆₋₁₀ arylamino(C₁₋₆)alkyl, C₆-10 aryloxy(C₁₋₆)alkyl, C₆-10 ar(C₁₋₆)alkylamino(C₁₋₆)alkyl, C₆₋₁₀ ar(C₁₋₆)alkyloxy(C₁₋₆)alkyl, C₆₋₁₀ ar(C₁₋₆ alkyl)carbonylamino(C₁₋₆)alkyl, C₆₋₁₀ ar(C₁₋₆ alkyl)carbonyloxy(C₁₋₆)alkyl, (C₆₋₁₀ aryl)carbonylamino(C₁₋₆)alkyl, (C₆₋₁₀ aryl)carbonyloxy(C₁₋₆)alkyl, (C₆₋₁₀ aryl)carbonyl(C₁₋₆)alkyl and C₆₋₁₀ ar(C₁₋₆ alkyl)carbonyl(C₁₋₆)alkyl, wherein the aryl portion of each of the preceding groups is optionally substituted with 1-4 substituents independently selected from the group consisting of halo, hydroxyl, C₁₋₆ alkyl, C₃₋₈ cycloalkyl, C₁₋₆ haloalkyl, C₁₋₆ hydroxyalkyl, C₂₋₆ alkenyl, C₂₋₆ alkynyl, C₁₋₆ alkoxy, (C₁₋₆ alkyl)carbonyl, (C₁₋₆ alkoxy)carbonyl, amino, amino(C₁₋₆)alkyl, aminocarbonyl, mono(C₁₋₆ alkyl)aminocarbonyl, di(C₁₋₆ alkyl)aminocarbonyl, C₁₋₆ alkylamino, di(C₁₋₆)alkylamino, (C₁₋₆ alkyl) carbonylamino, C₆₋₁₀ arylamino, (C₆₋₁₀ aryl)carbonylamino, mono(C₆₋₁₀ aryl) aminocarbonyl, di(C₆₋₁₀ aryl)aminocarbonyl, mono(C₆₋₁₀ ar(C₁₋₆ alkyl)) aminocarbonyl, di(C₆₋₁₀ ar(C₁₋₆ alkyl))aminocarbonyl, N-(C₆₋₁₀)aryl-N-(C₁₋₆ alkyl)aminocarbonyl, N-(C₆₋₁₀)ar(C₁₋₆)alkyl-N-(C₁₋₆ alkyl)aminocarbonyl, N-(C₆₋₁₀)ar(C₁₋₆)alkyl-N-(C₆₋₁₀ aryl)aminocarbonyl, C₁₋₆ alkylthio, C₆₋₁₀ arylthio, C₆₋₁₀ ar(C₁₋₆)alkylthio, carboxy, carboxy(C₁₋₆)alkyl, nitro, cyano, heteroaryl and saturated or partially unsaturated heterocycle, wherein the heteroaryl and saturated or partially unsaturated heterocycle are independently monocyclic or fused bicyclic and independently have 5 to 10 ring atoms, wherein one or more of the ring atoms are independently selected from the group consisting of oxygen, nitrogen and sulfur. Examples of modified nucleotide analogues, and of appropriate modification groups, are shown in FIG. 12-14.

To facilitate reiterative, abortive synthesis initiation events, the NTPs and/or NTP analogs may be added to the reaction mixture before and/or during the synthesis reaction include a chain terminator, which is capable of terminating the synthesis event initiated by the polymerase. Use of the chain terminator stalls the polymerase during the synthesis reaction, inhibits formation of a processive elongation complex, and thereby promotes the reiterative synthesis of short abortive oligonucleotides from the target site. (Daube and von Hippel, Science, 258: 1320-1324 (1992)).

In accordance with the invention, a chain terminator may comprise any compound, composition, complex, reactant, reaction condition, or process (including withholding a compound, reactant, or reaction condition) which is capable of inhibiting the continuation of transcription or replication by the polymerase during the primer extension reaction. In one embodiment, a suitable chain terminator is NTP deprivation, that is, chain termination results from depriving the polymerase of the particular NTP that corresponds to the subsequent complementary nucleotide of the template sequence. In other words, since NTP requirements for chain elongation are governed by the complementary strand sequence, given a defined template sequence and a defined primer length, a selected NTP may be withheld from the reaction mixture such that termination of chain elongation by the polymerase results when the reaction mixture fails to provide the polymerase with the NTP that is required to continue transcription or replication of the template sequence.

Alternatively, in another embodiment, the chain terminator may include nucleotide analogs which, upon incorporation into an oligonucleotide product by the polymerase, effect the termination of nucleotide polymerization.

Additional information concerning reaction conditions, nucleotides, and nucleotide molecules have been described in WO 03/038042 and U.S.

Application Publication Number US-2003-0099950.

Reaction Conditions

Appropriate reaction media and conditions for carrying out the methods of the present invention may include an aqueous buffer medium that is optimized for the particular polymerase, that may or may not be supplemented with various ions, buffers, chelating agents, polyanionic or cationic molecules, protein carriers or other proteins, including transcription factors (sigma, NusA, Rho, lysozyme, GreA, GreB, NusG, etc.). Variations in all of the reaction components potentially can alter the ratio of abortive transcripts to full-length transcripts. For example, a high molar ratio of RNA polymerase to template enhances the frequency of abortive transcription over full-length transcription on the lambda P_{R} promoter. This effect apparently arises from collisions between tandem polymerases at the promoter. Certain RNA polymerase mutants have elevated rates of abortive transcription compared to the wild-type polymerase. The relative level of abortive transcription is sensitive to the nucleotide sequence of the promoter. Any aspect of the methods of the present invention can occur at the same or varying temperatures, including isothermally, or in some embodiments, the temperature for the transcription or replication may differ from that temperature(s) used elsewhere in the assay.

In accordance with various aspects and embodiments of the invention, the target nucleic acid molecules may be hybridized to an oligonucleotide capture probe, a mononucleotide or oligonucleotide initiator which is complementary to a portion of the target nucleic acid, an APC linker sequence that is complementary to a portion of a target nucleic acid, and/or a target site probe that is complementary to regions on either side of the target site. Hybridization is conducted under conditions necessary to achieve the desired degree of specificity, using methods that are well-known to those skilled in the art. It may be necessary to denature the target nucleic acid in a test sample before hybridization.

Transcription conditions and reagents are well known in the art and have been described elsewhere. As described in Lu et al., U.S. Pat. No. 5,571,669, polymerase concentrations for transcription initiated from artificial transcription bubble complexes are generally about one order of magnitude higher than the ideal polymerase concentrations for promoter-initiated, or palindromic sequence-initiated, transcription.

In one embodiment, the components are added simultaneously at the initiation of the abortive synthesis and detection methods. In another embodiment, components are added in any order prior to or after appropriate timepoints during the method, as required and/or permitted by the various reactions. Such timepoints can be readily identified by a person of skill in the art. The various reactions in the methods of the invention can be stopped at various timepoints and then resumed at a later time. These timepoints can be readily identified by a person of skill in the art. Methods for stopping the reactions are known in the art, including, for example, cooling the reaction mixture to a temperature that inhibits enzyme activity.

Abortive Synthesis and Detection Methods of the Invention

In accordance with an aspect of the invention, detectable oligonucleotide products are synthesized from a target nucleic acid template. The oligonucleotide products may also include label moieties on the initiator and/or on the NTPs or NTP analogs that are incorporated by the polymerase into each oligonucleotide product that is synthesized on the target nucleic acid and/or on other molecules which are part of the synthetic complex or which interact with one or more components of the synthetic complex.

In accordance with the invention, detection of the oligonucleotide products is indicative of the presence of the target sequence. In some embodiments, the target sequence comprises the target molecule of interest. In other embodiments, the target molecule of interest is distinct from the target sequence from which oligonucleotides products are produced; thus the detection of oligonucleotides indicates presence of the target sequence which indicates presence of the target molecule of interest. In these embodiments the molecule of interest may be a protein, peptide, hapten, carbohydrate, toxin, lipid, ion, or another nucleic acid, for example. Thus, in some embodiments, the presence of the target sequence may further indicate the presence of a protein of interest. In some embodiments, an abortive promoter cassette is coupled to a DNA, RNA, PNA (protein nucleic acid, wherein the nucleotide backbone is replaced with amino-group linkages, rather than phosphate linkages), antibody, binding protein, hapten, signalling protein, ion or other molecule with specificity for the molecule of interest.

Quantitative analysis is also feasible. Direct and indirect detection methods (including quantitation) are well known in the art. For example, by comparing the amount of oligonucleotide products that are generated from a test sample containing an unknown amount of a target nucleic acid to an amount of oligonucleotide products that were generated from a reference sample that has a known quantity of a target nucleic acid, the amount of a target nucleic acid in the test sample can be determined. The reiterative abortive synthesis initiation and detection methods of the present invention can also be extended to the analysis of genetic sequence alterations in the target nucleic acid, as further described below.

The following examples of the abortive synthesis and detection methods of the invention are provided to more specifically describe the invention. These exemplary methods are intended to be merely illustrative and are not intended to limit the description provided above. It will be appreciated that various other embodiments may be practiced, given the above general description. For example, reference to the use of a primer means that any of the primers described herein may be used, including RNA initiators.

In accordance with an aspect of the invention, a method for detecting the presence of a target polynucleotide by generating multiple detectable oligonucleotide products through reiterative synthesis initiation events on the target polynucleotide is provided.

FIG. 2 diagrammatically illustrates the various reactants that may be combined and reacted in the presence of RNA polymerase to synthesize multiple detectable oligonucleotide products. The methods of the invention may be performed using a test sample that potentially contains a target sequence. The test sequence may be detected directly or the product of primer-extension or reverse transcription of the target may be detected.

Sequences or tags may be added to the copy of the target (e.g., biotin, ssDNA regions). The test sample may include double-stranded DNA, single-stranded DNA, or RNA. The DNA or RNA may be isolated and purified by standard techniques for isolating DNA or RNA from cellular, tissue, or other samples.

In one embodiment, the target sequence is immobilized by a sequence-specific (e.g., gene-specific) oligonucleotide capture probe that is attached to a solid matrix, such as a microtiter plate. The immobilized capture probe is treated under hybridizing conditions with a test sample that includes single-stranded DNA (i.e., denatured DNA) or RNA. Any target sequence that is present in the test sample hybridizes to the capture probe and is then exposed to additional reagents in accordance with the invention.

In one embodiment, an initiator (n 5′—R₁—(N₁)ₓ—OH 3′) hybridizes with the target sequence upstream of a target site in the presence of the target site probe (FIG. 4) and facilitates catalysis of a polymerization reaction at the target site by the polymerase. The initiation primer may be comprised of nucleosides, nucleoside analogs, nucleotides, and nucleotide analogs, and may vary in the number of nucleotides, as described in WO 03/038042 and U.S. Application Publication Number US-2003-0099950. A suitable RNA polymerase is employed to synthesize an oligoribonucleotide product from the target sequence or any portion thereof.

During the polymerization reaction, the initiator is extended or elongated by the polymerase through the incorporation of nucleotides which have been added to the reaction mixture. As the polymerase reaction proceeds, the polymerase extends the initiator, as directed by the template sequence, by incorporating corresponding nucleotides that are present in the reaction mixture. In one embodiment, these reactant nucleotides comprise a chain terminator (e.g., n 5′ pppN_{T}—R₂, a chain terminating nucleotide analog, as described above). When the polymerase incorporates a chain terminator into the nascent oligonucleotide product, chain elongation terminates due to the polymerase's inability to catalyze the addition of a nucleotide at the 3′ position on the pentose ring of the chain terminator. Consequently, the polymerase aborts the initiated synthesis event by releasing the oligonucleotide product (i.e., 5′ R₁—(N_{I})_{z}pN_{T}—R₂, where z=x+y) and reinitiating the abortive initiation synthesis reaction at the target site.

The abortive initiation reaction may be controlled such that the polymerase aborts synthesis after extending the initiator by a predetermined number of nucleotides. For example, if it is desirable to terminate the synthesis reaction after the initiator has been extended by a single nucleotide, this may be accomplished by, for example, either: (1) adding to the reaction mixture only nucleotides that are chain terminators, thereby inhibiting polymerization after the first nucleotide is incorporated by the polymerase; or (2) if the genetic sequence of the target site is known, adding to the reaction mixture only a preselected chain terminating nucleotide analog (i.e., nucleotide analogs which comprise one of A, G, T, C, or U) that is complementary to the nucleotide at the target site. Alternatively, if it is desirable to terminate the synthesis reaction after the initiator has been elongated by a predetermined number of nucleotides, and if the genetic sequence of the target site is known, this may be accomplished by, for example, adding to the reaction mixture a preselected chain terminating nucleotide analog (i.e., nucleotide analogs which comprise one of A, G, T, C, or U) that is complementary to an Nth nucleotide from the target site, where N is the predetermined number of nucleotides comprised by the oligonucleotide product, exclusive of the initiator. In this manner, multiple abortive oligonucleotide products that comprise the initiator and a chain terminating nucleotide analog are synthesized by the polymerase.

The polymerase releases the oligonucleotide product without translocating from the enzyme binding site or dissociating from the target polynucleotide sequence. Nucleotide deprivation can be used to sequester the polymerase at the polymerase binding site. For example, if only an initiator and a terminator are supplied, elongation by the polymerase will not be possible.

Furthermore, reaction conditions may be optimized for abortive transcription initiation, whereby it is favorable for the polymerase to remain bound to the polymerase binding site even in the presence of elongating nucleotides. The abortive initiation reaction buffer will be optimized to increase the abortive events by adjusting the concentrations of the salts, the divalent cations, the glycerol content, and the amount and type of reducing agent to be used. In addition, “roadblock” proteins may be used to prevent the polymerase from translocating.

In another aspect of the invention, as diagrammatically illustrated in FIG. 4, a target site probe may be used to form a bubble complex in a target region of the target sequence. As described above, the bubble complex comprises double-stranded regions that flank a single-stranded region that includes a target site. In this embodiment, the target site probe is used to direct the polymerase to the target site by positioning the target site at the junction of the single-stranded bubble region and a downstream duplex region on the target sequence. The polymerase associates with an initiator and initiates a synthesis reaction at the target site on the template sequence. The polymerase elongates the initiator to synthesize an abortive oligonucleotide product through the incorporation of nucleotides, which comprise a suitable chain terminator. Both the initiator and the nucleotides, including the chain terminating nucleotide, may be modified with a label moiety.

An illustrative procedure for detecting multiple oligonucleotide products through reiterative synthesis initiation events on a target sequence, therefore, may include: (a) immobilizing an oligonucleotide capture probe which is designed to hybridize with a specific or general target sequence; (b) hybridizing the oligonucleotide capture probe with a test sample which potentially contains a target sequence; (c) hybridizing the target sequence with a target site probe; (d) modifying at least one of an initiator and nucleotides comprising a chain terminator to enable detection of the oligonucleotide product synthesized by the polymerase; (e) hybridizing the target sequence with the primer; and (f) extending the initiator with a polymerase such that the polymerase reiteratively synthesizes an oligonucleotide product that is complementary to a target site by incorporating complementary nucleotides comprising a chain terminator and releasing an abortive oligonucleotide product without either translocating from an enzyme binding site or dissociating from the target sequence.

During transcription of the template by the RNA polymerase, the RNA initiator is extended by the RNA polymerase through the incorporation of nucleotides that have been added to the reaction mixture. As the polymerase reaction proceeds, the RNA polymerase extends the RNA initiator, as directed by the template sequence, by incorporating corresponding nucleotides that are present in the reaction mixture. In one embodiment, these reactant nucleotides comprise a chain terminator (e.g., n 5′ pppN_{T}—R₂, a chain terminating nucleotide analog, as described above). When the RNA polymerase incorporates a chain terminator into the nascent transcript, chain elongation terminates due to the polymerase's inability to catalyze the addition of a nucleotide at the 3′ position on the ribose ring of the chain terminator, and the RNA polymerase aborts the initiated transcription event by releasing the transcript and reinitiating transcription at the target site. The abortive transcription initiation reaction may be controlled such that multiple abortive oligonucleotide transcripts of a predetermined length and comprising the RNA primer and a chain terminating nucleotide analog are generated.

In an exemplary embodiment, the RNA initiator may be a mononucleotide and the nucleotides provided in the reaction mixture may comprise solely chain terminators. In this embodiment, transcription is aborted by the RNA polymerase after the RNA initiator has been extended by a single nucleotide and an abortive dinucleotide transcript is generated. In another embodiment, the RNA initiator may comprise a dinucleotide or a trinucleotide, for example, and an abortive transcription initiation event may generate an abortive transcript comprising a trinucleotide or a tetranucleotide, respectively. It will be appreciated that abortive transcripts of any desired length may be obtained, depending upon the length of the RNA initiator and the nature and composition of the reactant nucleotides that are selected for inclusion in the reaction mixture. For example, if the nucleotide sequence of the template is known, the components (e.g., target site, initiator, and reactant nucleotides) of the transcription reaction may be selected such that abortive transcripts of any desired length are generated by the method of the invention.

In another aspect of the invention, the RNA initiator includes a moiety (e.g., R₁, as depicted in FIG. 3) which may be covalently bound to the 5′ phosphate group, the 2′ position of the ribose ring, or the purine or pyrimidine base of one of the nucleotides or nucleotide analogs that are incorporated into the RNA initiator. Additionally, the reactant nucleotides and/or nucleotide analogs that are included in the reaction mixture for incorporation into the oligonucleotide transcript by the RNA polymerase may each also include a moiety (e.g., R₂, as depicted in FIG. 3), which is covalently bonded to either the nucleobase or the 2′ position or 3′ position of the ribose ring. The moieties R₁ and R₂ may each comprise H, OH, or any suitable label moiety, reporter group, or reporter group precursor, as described in greater detail above.

An illustrative procedure for detecting multiple oligonucleotide transcripts through reiterative transcription initiation events on a target sequence, therefore, may include: (a) optionally immobilizing an oligonucleotide capture probe which is designed to hybridize with a specific or general target sequence; (b) optionally hybridizing the oligonucleotide capture probe with a test sample which potentially contains a target sequence; (c) optionally hybridizing the target sequence with a target site probe; (d) modifying at least one of an RNA initiator and nucleotides comprising a chain terminator to enable detection of the oligonucleotide transcript synthesized by the RNA polymerase; (e) hybridizing the target sequence with the RNA initiator; and (f) extending the RNA initiator with an RNA polymerase such that the RNA polymerase reiteratively synthesizes an oligonucleotide transcript that is complementary to a target site by incorporating complementary nucleotides comprising a chain terminator and releasing an abortive oligonucleotide transcript without substantially translocating from the polymerase binding site or dissociating from the target sequence.

The multiple reiterative oligonucleotide transcripts may be rapidly detected and quantified with mass spectrometry. For example, as shown in Tables 1a and 1b, Example 2, mass spectrometry can distinguish between any trinucleotide. Differences between trinucleotides may be accentuated by the use of labels, including metal ions, isotopes, or other molecules. The integrated area under the peak corresponding to the trinucleotide will indicate the amount (in either absolute or relative terms) of the trinucleotide. Finally, the sample may be analyzed almost in real time. Thus, a sample may be subject to abortive transcription and in close to real time, the results read indicating the presence of a target molecule, its abundance and, by determining which species of oligonucleotide transcript is/are produced, the nature of the target molecule.

Multiplexing

The methods of the present invention are not limited to a single type of abortive transcription reaction at one time, but also include simultaneously conducting multiple reactions at one time, i.e. multiplexing. In other words, multiple different abscription reactions may occur simultaneously on a single sample.

Multiplexing has a number of advantages. The presence of multiple detectable signals increases sensitivity. Specificity is also increased by the use of multiple detectable signals. For example, if 4 probes are used, 4 different abortive transcription products will be obtained. Detecting the presence of 4 abscription products will confirm the presence of the target molecule of interest, whereas the detection of only 1 abscription product may be regarded as a false positive reaction.

In one embodiment, multiplexing is performed on the same target molecule of interest. In one embodiment multiple TSPs are used, each of which are specific for a different section of DNA from the target nucleic acid. In another embodiment, different DNA probes, each of which is attached to a different APC, or APC-dendrimer complex, are allowed to hybridize to target nucleic acid sequence. In another embodiment, different antibodies are used, each of which is attached to a different APC or APC-dendrimer complex. When the abscription reaction is performed it will generate as many types of signals as exist different types of APC. The resulting multiplex signal is examined, by mass spectroscopy, for example.

In some embodiments, multiple distinct reactions are performed on a sample comprising a mixed population of target molecules of interest. In one embodiment, the sample may comprise multiple pathogens of interest, each of which may be detected by an individual TSP specific for a section of DNA unique to each pathogen. Alternatively, the pathogens may be detected by an APC (or APC-dendrimer complex) linked to a nucleic acid probe or antibody specific for a sequence or antigen from the target pathogen. By multiplexing, a range of different target molecules may be detected simultaneously, at an increased speed and lower cost than would be obtained by detection of each target molecule individually.

In some further embodiments, a multiplex reaction may comprise the detection of multiple target molecules, each target molecule being detected by one or more abortive transcription reaction.

Further Signal Amplification.

The products of abortive reiterative transcription may be detected directly or indirectly. In one embodiment, the products are subject to further direct amplification. In another embodiment, the product is introduced into a signal amplification cascade, increasing the sensitivity of detecting the target molecule.

In one signal cascade embodiment, abortive oligonucleotide transcripts are labelled with biotin; such biotin-labelled molecules may then be detected with avidin coupled to an enzyme that produces a detectable signal (such as HRP), for example. In another embodiment, avidin may be coupled to an APC or APC-dendrimer complex, from which may be synthesized further abortive oligonucleotide transcripts.

In another embodiment, the abortive oligonucleotide transcripts are used as reagents in a subsequent reaction, such as initiators in a transcription reaction, or as primers for a PCR reaction.

Applications of Abortive Synthesis and Detection Methods

The methods of the present invention can be used in a variety of diagnostic and analytical contexts including, but not limited to, assessing the methylation state of specific genes, detecting the presence of known genetic mutations, detecting the presence of pathogenic organisms, detecting mRNA expression levels, and detecting proteins. Mass Spectrometry is used to detect the reiterative oligonucleotide transcripts, rather than via flourescence, radioisotope or other method. Methods leading to the production of reiterative oligonucleotide transcripts and the uses thereof are only summarized herein for illustrative and nonlimiting purposes. Additional details relating to such methods may be found in WO 03/038042 and U.S. Application Publication Number US-2003-0099950.

DNA Methylation

The methods of the present invention may be used in diagnostic assays which detect epigenetic changes associated with disease initiation and progression by assessing the methylation state of specific genes and their regulatory regions that are known to be associated with particular disease-states. DNA methylation is a cellular mechanism for altering the properties of DNA without altering the coding function of that sequence. CpG methylation is potentially a powerful marker for cancer progression, and may also be associated with gene imprinting, the inactivation of transposable elements, and the inactivation of the X-chromosome in females. DNA methylation in the human genome is most frequent on Cs in the dinucleotide sequence CpG.

Thus, in one embodiment, the methods of the invention may be utilized to monitor disease initiation, progression, metastasis, recurrence, and any responses to treatment therapies by providing diagnostic techniques, which can detect altered methylation states and patterns. Methylated cytosine residues in a DNA fragment can be detected based upon the resistance of such residues to deamination by a deaminating agent, such as sodium bisulfite for example. As illustrated in FIG. 5, when denatured (i.e., single-stranded) DNA is exposed to a deaminating agent, such as sodium bisulfite, unmethylated cytosine (C) residues are converted into uracil residues (U), while methylated cytosine residues (5-mCyt) remain unchanged, thus changing their complementary base-pairing partner from guanine (G) to adenosine (A). However, the methylated cytosines (5-mCyt) retain their base-pairing specificity for G. In view of the foregoing, the level of methylation of the CpG islands in a target DNA sequence may be determined by measuring the relative level of unaltered CpG sites.

In another embodiment, as diagrammatically illustrated in FIG. 6, after the target DNA sequence has been deaminated, such as by treating the target DNA sequence with sodium bisulfite for example, a target site probe may be used to form a bubble complex that comprises a target CpG site on the target DNA sequence. In this embodiment, the target site probe is used to direct the RNA polymerase to the target CpG site by positioning the target CpG site at the junction of a single-stranded bubble region and a downstream duplex region on the target DNA sequence. In the illustrated embodiment, the target site probe comprises about 18-54 nucleotides: a first region which hybridizes to the target DNA sequence upstream of the target site comprises about 5-20 nucleotides; an internal second region of non-base-paired nucleotides comprises about 8-14 nucleotides; and a third region which hybridizes to the target DNA sequence downstream of the target site comprises about 5-20 nucleotides. The target site probe may be hybridized to the target DNA sequence either before or while the DNA target sequence is incubated with an RNA polymerase and a suitable RNA initiator. The polymerase associates with the RNA initiator and initiates transcription and RNA synthesis at the CpG site on the DNA template. The polymerase extends the initiator to synthesize an abortive oligonucleotide transcript through the incorporation of a suitable chain terminator. Either or both of the initiator and a chain terminating nucleotide may be modified.

In another embodiment, capture probes may be designed to capture the genes of interest, and abortive transcription initiation used to determine the methylation status of the desired genes. Each gene of interest could be removed from the sample by hybridization to a capture sequence, which is unique for the gene of interest.

Genetic Mutations

In another aspect of the invention, the methods disclosed herein may be used in diagnostic assays which detect mutations in the form of gross chromosomal rearrangements or single or multiple nucleotide alterations, substitutions, insertions, or deletions.

Pathogenic Organisms

In another aspect of the invention, the methods disclosed herein may be used in diagnostic assays which detect the presence of a particular nucleic acid (DNA or RNA), thereby serving to indicate the presence of either a particular or a generic organism which contains the gene, or which permit genetic typing of a particular organism without the need for culturing the organism. The test sample may be suspected of containing a target nucleic acid sequence from a particular microorganism, such as bacteria, yeast, viruses, viroids, molds, fungi, and the like. The test sample may collected from a variety of sources including but not limited to, animal, plant or human tissue, blood, saliva, semen, urine, sera, cerebral or spinal fluid, pleural fluid, lymph, sputum, fluid from breast lavage, mucusoal secretions, animal solids, stool, cultures of microorganisms, liquid and solid food and feed-products, waste, cosmetics, air, and water.

In some embodiments an oligonucleotide capture probe that is sequence-specific for a target pathogen polynucleotide is attached to a solid matrix. A target pathogen polynucleotide that is present in the test sample hybridizes to the capture probe, and washing is then performed to remove any components of the test sample that were not immobilized by the capture probe. Target DNA or RNA may be retrieved by addition of specific sequences via primer extension, for example. In one embodiment, the captured target pathogen polynucleotide is hybridized with an abortive promoter cassette (APC). The APC linker sequence includes a single-stranded overhang region on either its 3′ or 5′ end (depending upon the orientation needed to create an antiparallel hybrid with the capture probe). In other words, the APC linker is complementary to the sequence on the free end of the captured target pathogen polynucleotide, thereby permitting the APC linker to hybridize to the target pathogen polynucleotide. Abortive transcription is then peformed as described herein, generating reiterative oligonucleotide transcripts which are then detected with Mass Spectrometry.

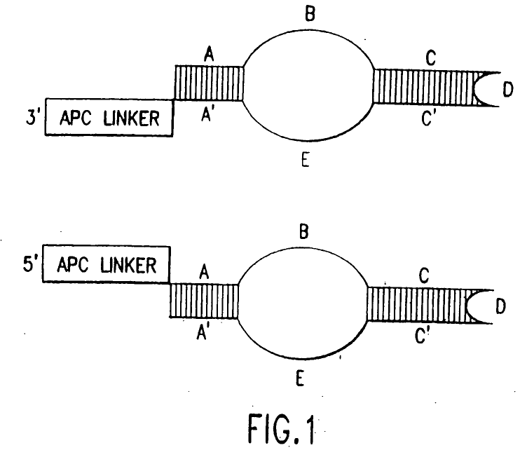

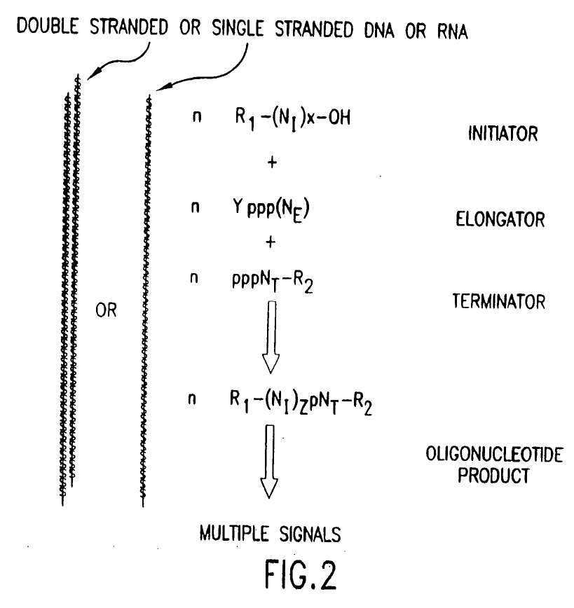

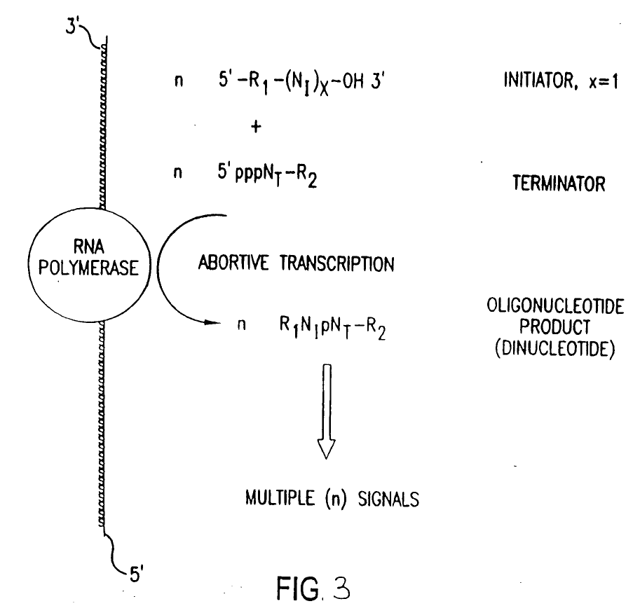

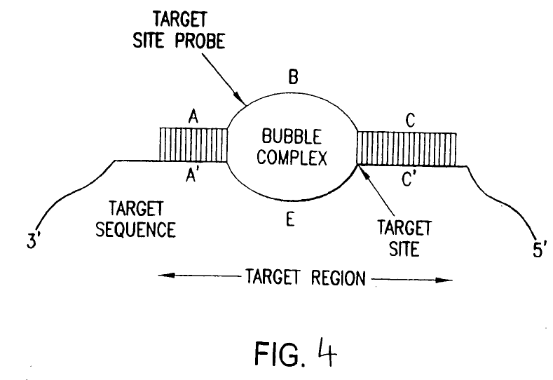

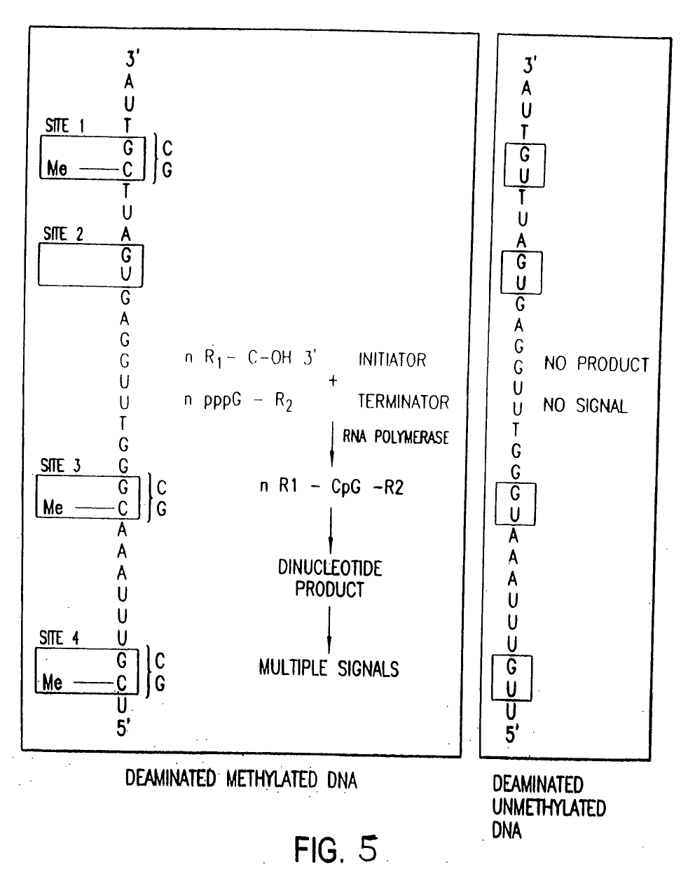

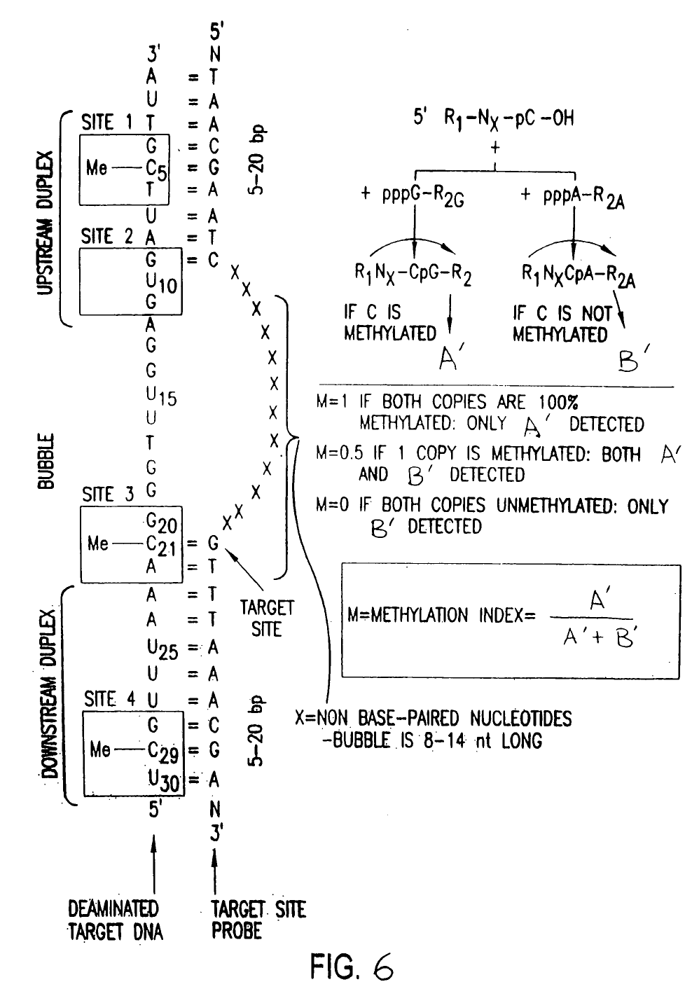

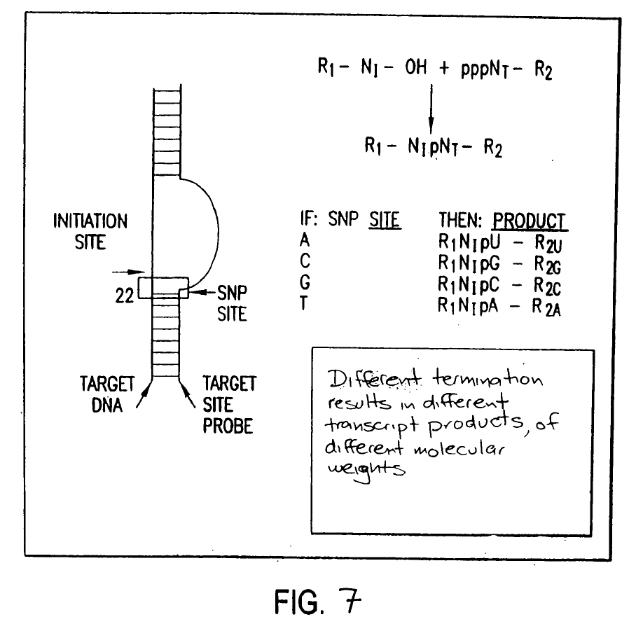

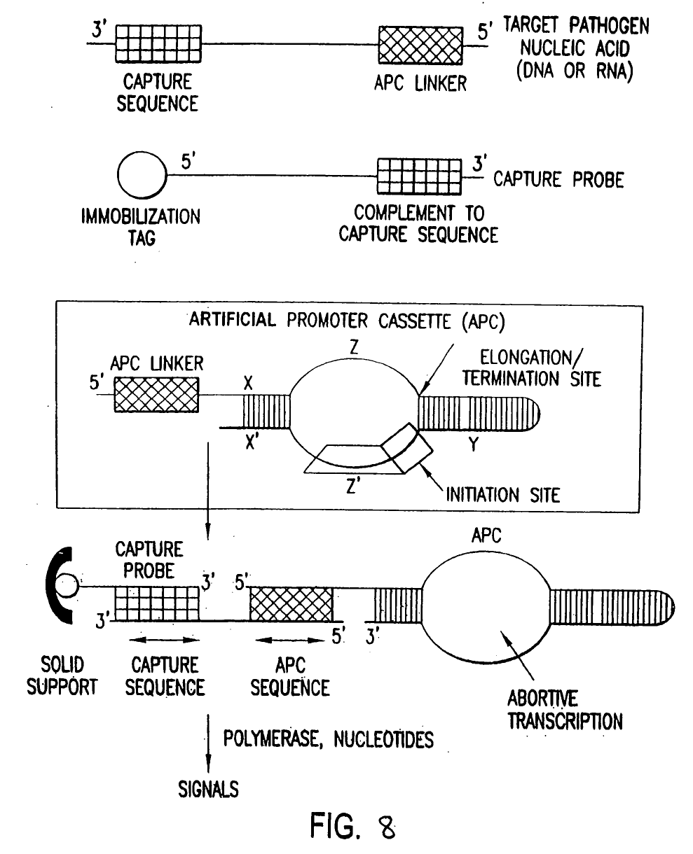

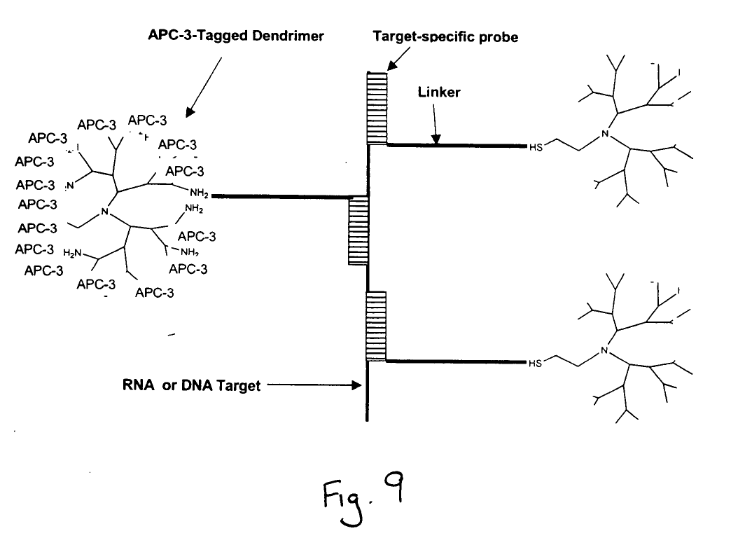

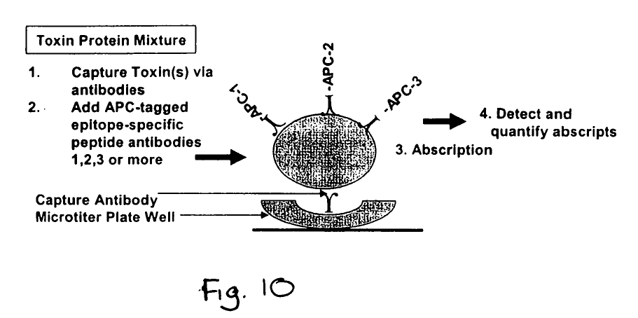

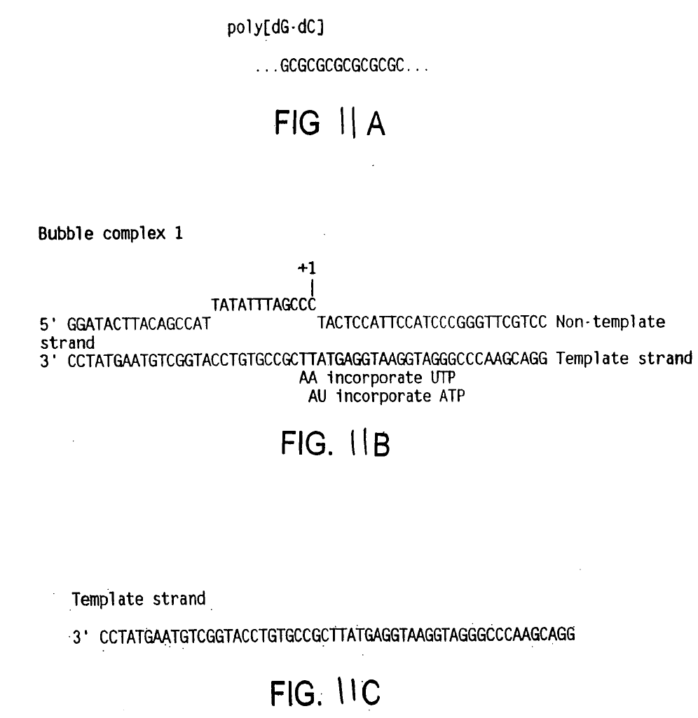

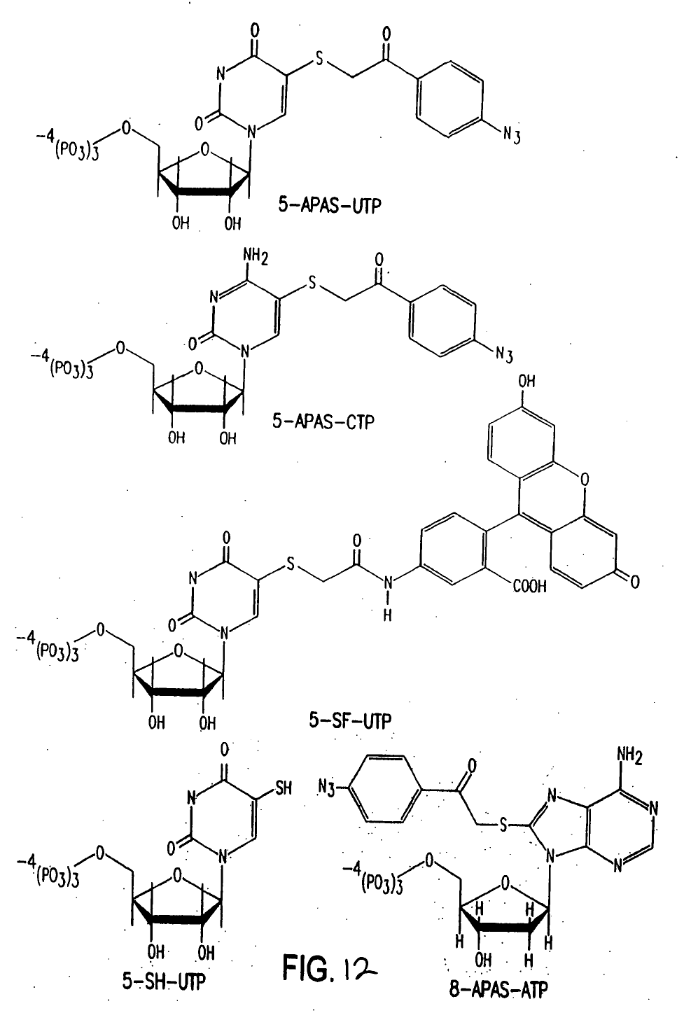

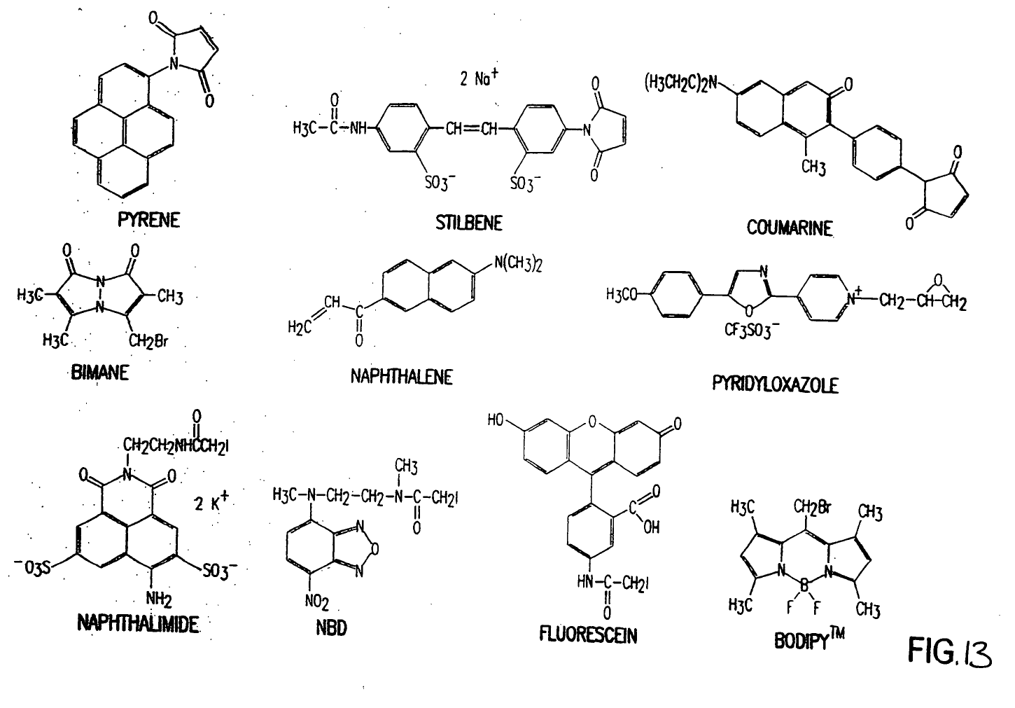

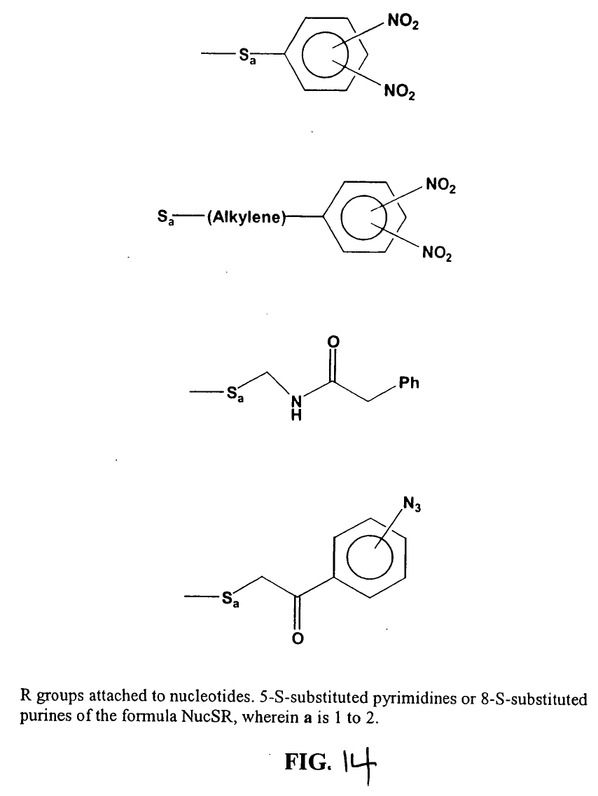

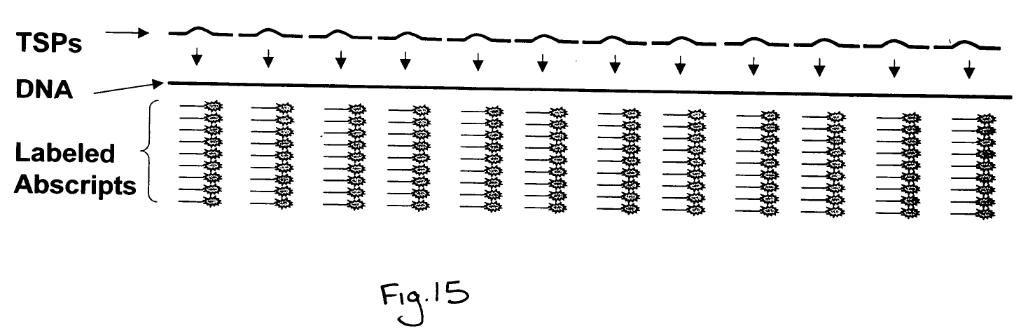

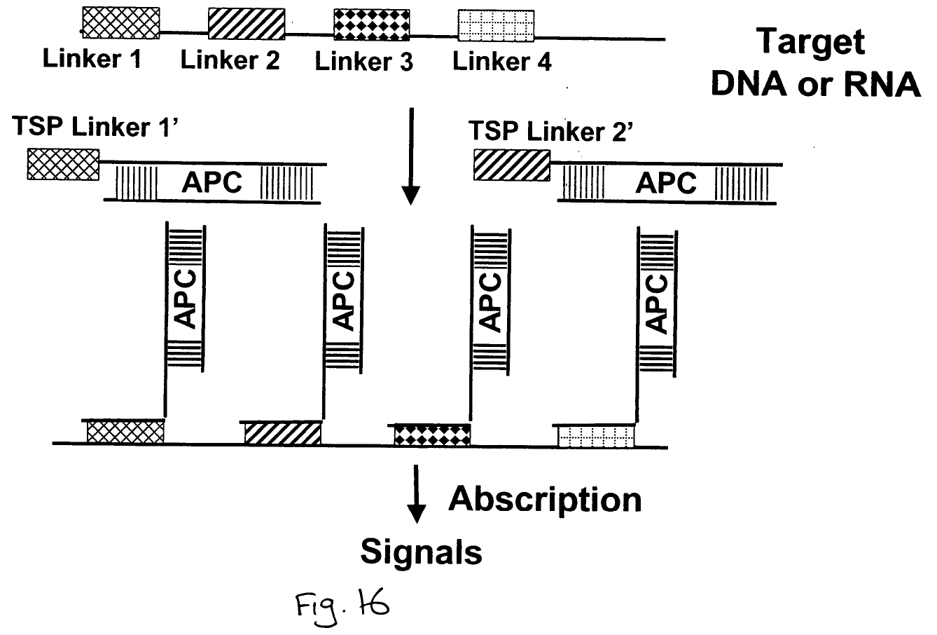

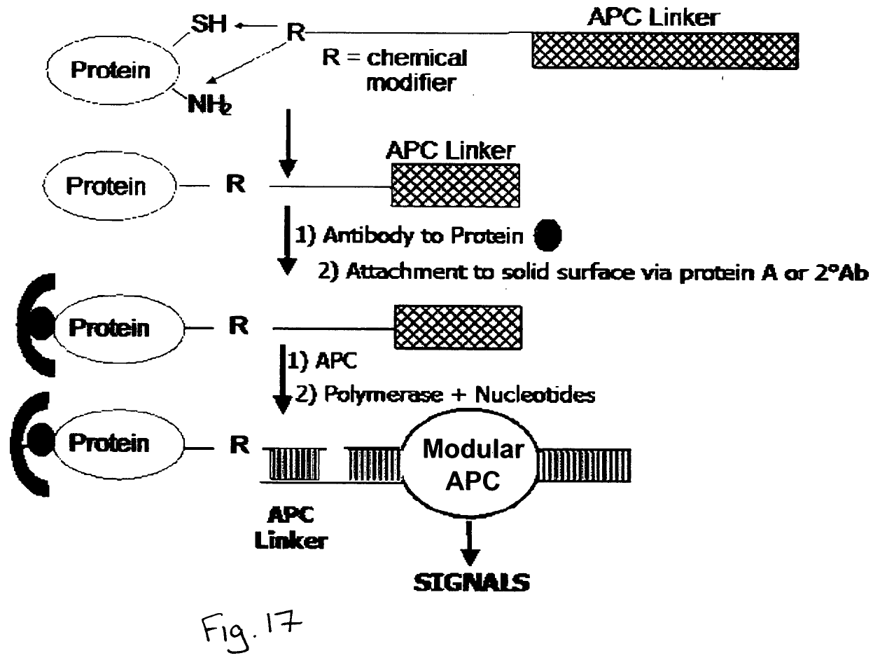

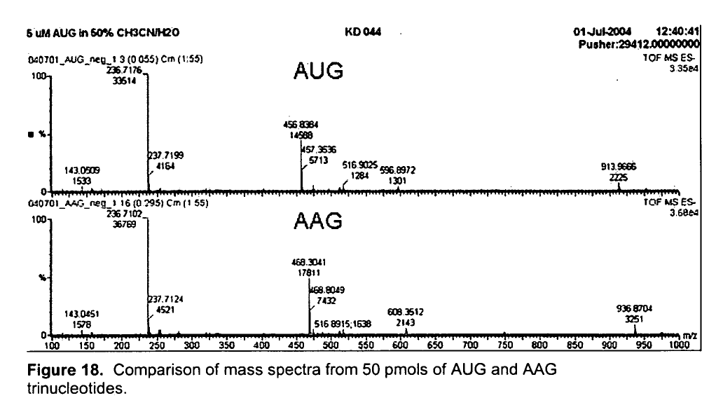

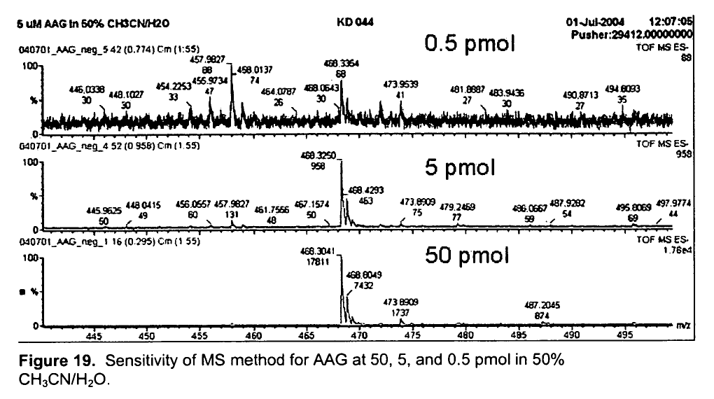

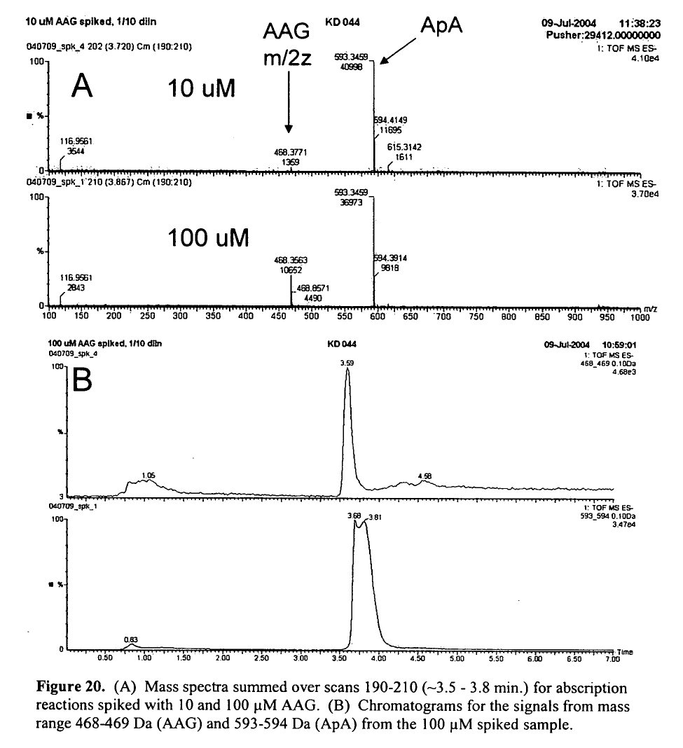

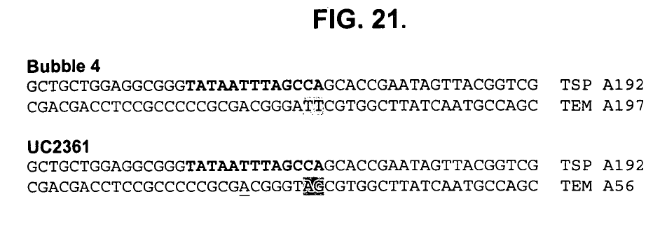

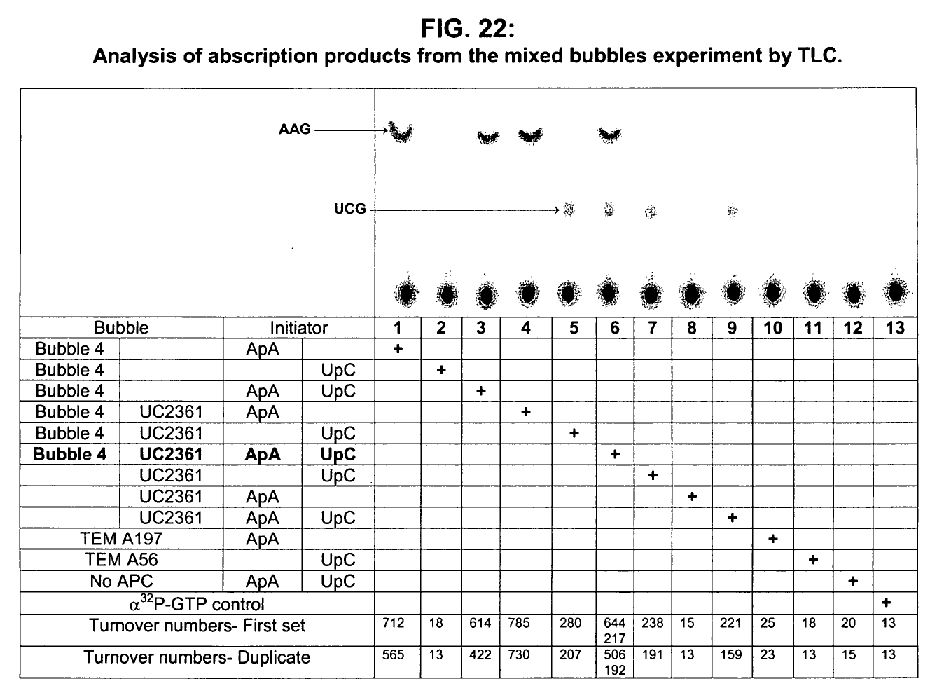

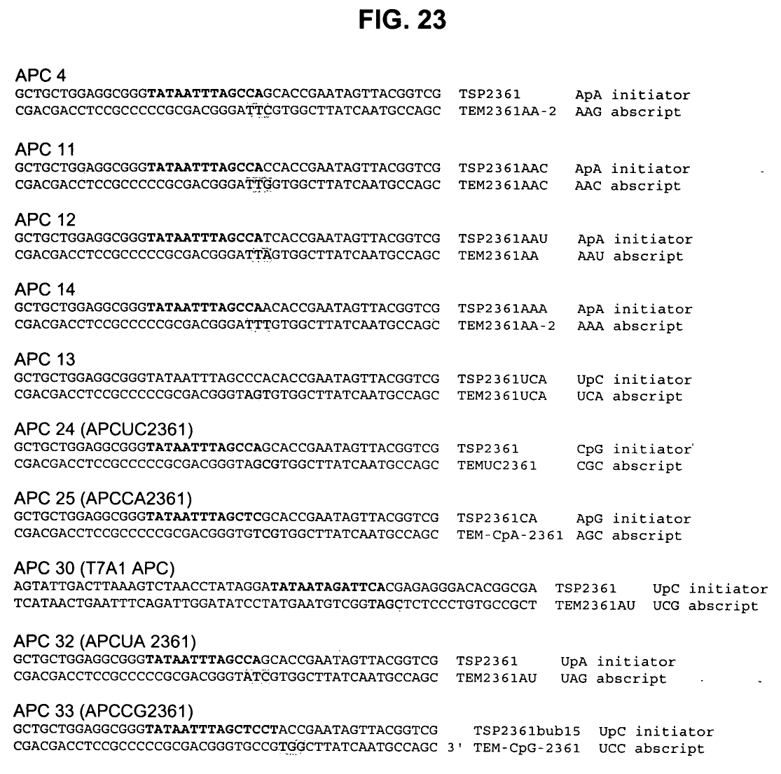

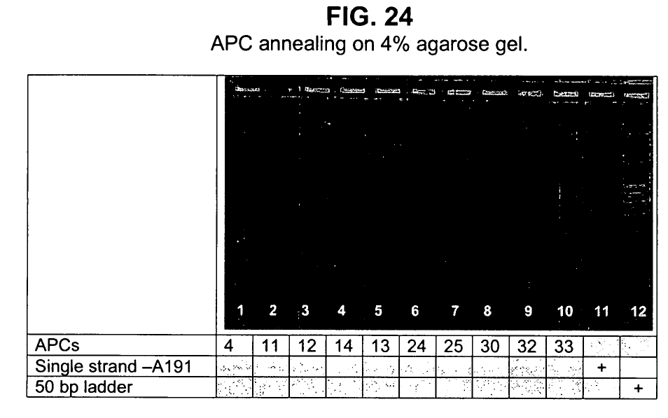

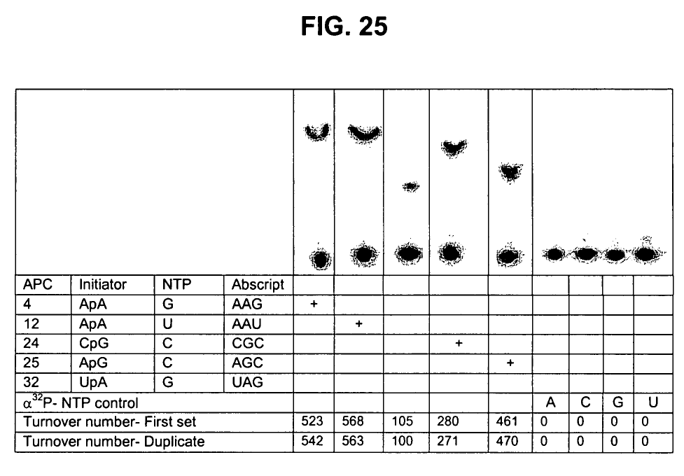

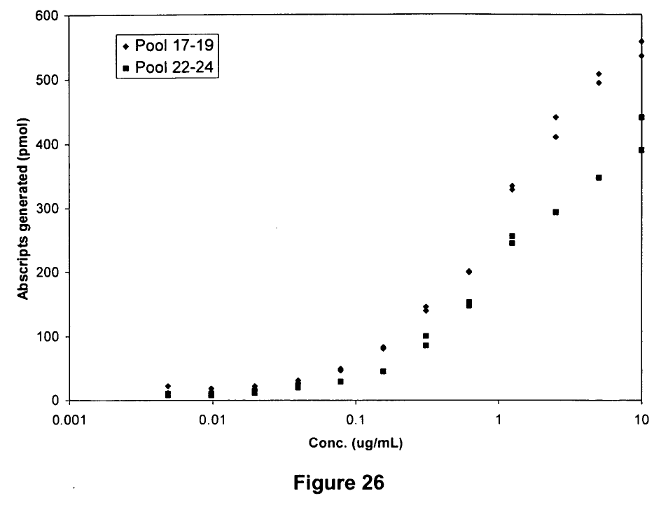

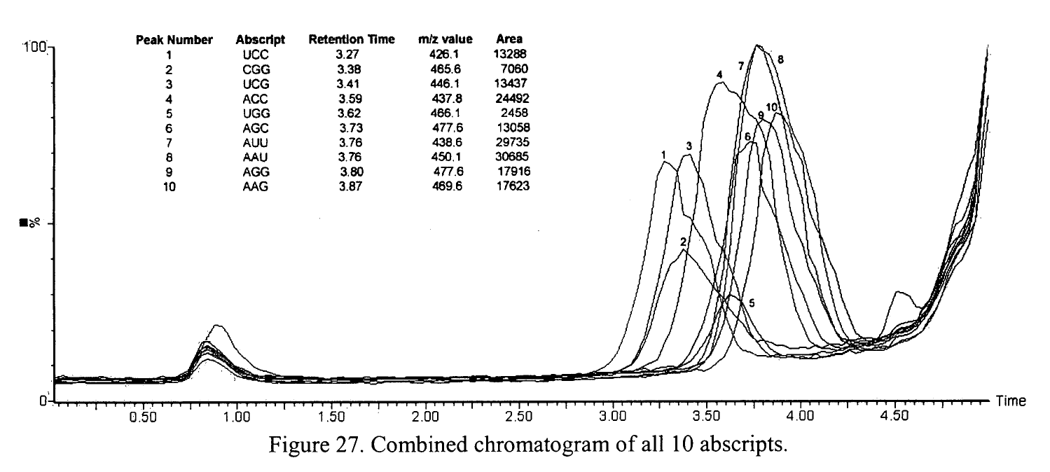

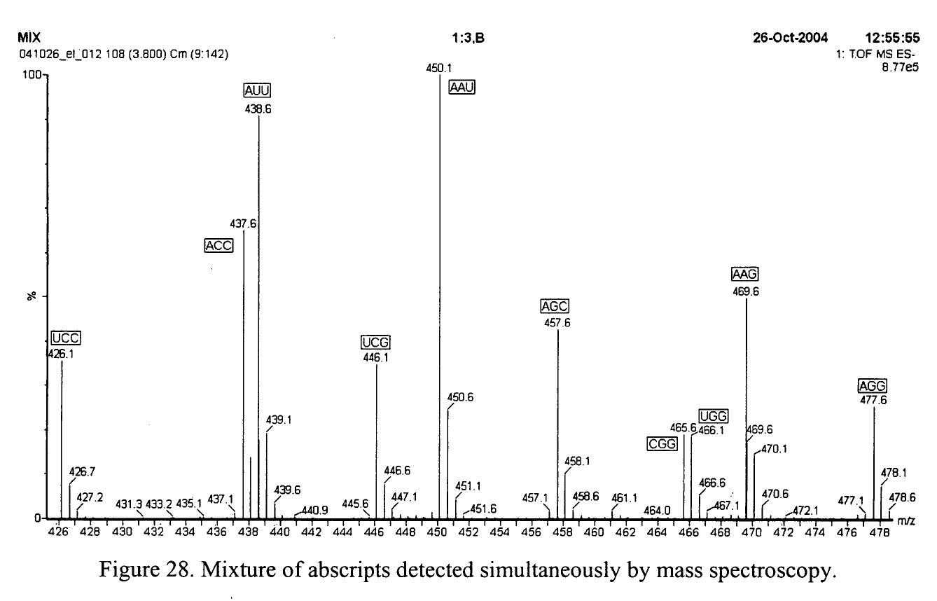

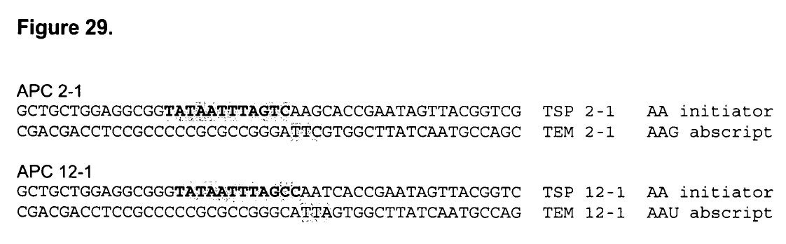

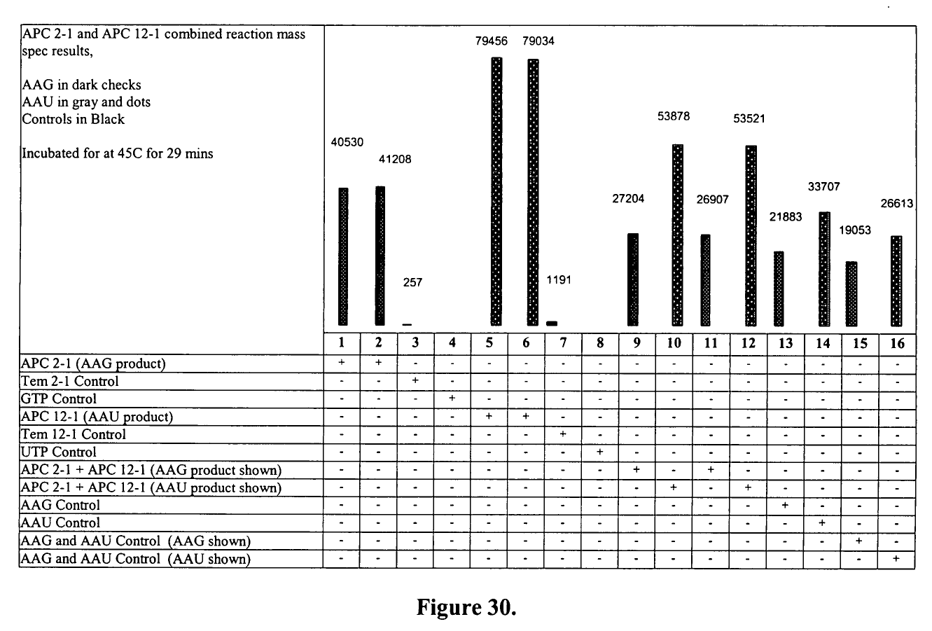

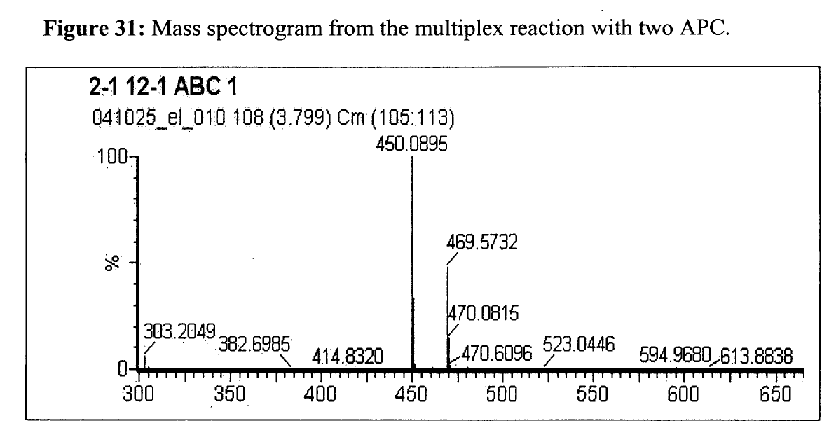

An illustrative procedure for detecting the presence of pathogens (FIG. 8), therefore, may include: (a) optionally immobilizing a capture probe designed to hybridize with a target pathogen polynucleotide; (b) optionally hybridizing the capture probe with a test sample which potentially contains a target pathogen polynucleotide. The target nucleic acid may be copied to DNA via reverse transcription (for RNA pathogens) or primer extension (for DNA pathogens). In both bases, a DNA sequence corresponding to the Abortive Promoter Cassette (APC) linker will be added to the target copy (FIG. 1); (c) optionally washing the captured target pathogen polynucleotide to remove any unhybridized components of the test sample; (d) hybridizing the captured target pathogen polynucleotide with an abortive promoter cassette; (e) modifying at least one of a initiator and nucleotides comprising a chain terminator to enable detection of the oligonucleotide product synthesized by the polymerase; (f) hybridizing the abortive promoter cassette with a initiator; (g) extending the initiator with a polymerase such that the polymerase reiteratively synthesizes an oligonucleotide product that is complementary to a target site by incorporating complementary nucleotides comprising a chain terminator and releasing an abortive oligonucleotide product without either translocating from an enzyme binding site or dissociating from the APC; and (h) detecting and optionally quantifying the multiple abortive oligonucleotide products using mass spectrometry

In another aspect, the invention provides a method for detecting the presence of pathogens in a test sample using mass spectrometry. The method comprises: (a) attaching a target pathogen polynucleotide or protein in said test sample to an artificial promoter cassette comprising a region that can be detected by transcription by a polymerase; (b) incubating said APC-tagged target molecule with an initiator, an RNA-polymerase, an elongator and/or terminator; (c) synthesizing an oligonucleotide transcript that is complementary to the initiation start site of the APC, wherein said initiator is extended until transcription terminates and the oligonucleotide is released, thereby synthesizing multiple reiterative oligonucleotide transcripts; and (d) determining the presence of a pathogen by detecting or quantifying said reiteratively synthesized oligonucleotide transcripts synthesized from said test sample using mass spectrometry.

In still a further aspect, the invention provides a method for detecting pathogens in a test sample using a capture probe and mass spectrometry. The method comprises: (a) immobilizing a capture probe designed to interact with a target DNA or RNA polynucleotide in said test sample; (b) mixing said capture probe with a test sample that potentially contains said target polynucleotide; (c) attaching target polynucleotide in said test sample to an artificial promoter cassette comprising a region that interacts with the target pathogen polynucleotide, and a region that can be detected by transcription by a polymerase; (d) incubating said target polynucleotide with an RNA-polymerase, initiator, an elongator and/or terminator; (e) synthesizing an oligonucleotide transcript that is complementary to said initiation transcription start site of APC, wherein said initiator is extended until transcription terminates and the oligonucleotide is released thereby synthesizing multiple reiterative oligonucleotide transcripts; and (f) determining the presence or absence of a pathogen by detecting or quantifying said reiteratively synthesized oligonucleotide transcripts using mass spectrometry.

The methods of the invention are particularly useful for monitoring the presence or absence of pathogenic nucleic acids and proteins. The invention can be used to detect, diagnose, and monitor diseases, and/or disorders associated with pathogenic polypeptides or polynucleotides. The invention provides for the detection of the aberrant expression of a polypeptide or polynucleotide. The method comprises (a) assaying the expression of the polypeptide or polynucleotide of interest in cells, tissue or body fluid of an individual using the methods of abortive initiation transcription described above, and (b) comparing the level of gene expression, protein expression, or presence of sequences of interest with a standard gene or protein expression level or seqeunce of interest, whereby an increase or decrease in the assayed polypeptide or polynucleotide level compared to the standard level is indicative of aberrant expression indicating presence of a pathogen of interest.

The presence of an abnormal amount of transcript in biopsied tissue or body fluid from an individual may provide a means for detecting the disease prior to the appearance of actual clinical symptoms. A more definitive diagnosis of this type may allow health professionals to employ preventative measures or aggressive treatment earlier thereby preventing the development or further progression of the disease caused by the pathogen.

The invention is particularly useful for monitoring the presence of pathogenic organisms including but not limited to E. coli, Steptococcus, Bacillus, Mycobacterium, HIV, and Hepatitis.

The methods of the invention may be used to test for pathogenic microorganisms in aqueous fluids, in particular water (such as drinking water or swimming or bathing water), or other aqueous solutions (such as fermentation broths and solutions used in cell culture), or gases and mixtures of gases such as breathable air, and gases used to sparge, purge, or remove particulate matter from surfaces. Breathable air from any source including but not limited to homes, schools, classrooms, workplaces, aircraft, spacecraft, cars, trains, buses, and any other building or structure where people gather, may be tested for the presence of pathogenic microorganisms.

mRNA Expression

In another aspect of the invention, the methods disclosed herein may be used in diagnostic assays which detect messenger RNA (mRNA) expression levels in a quantitative or non-quantitative manner.

Protein Detection

In another aspect of the invention, the methods disclosed herein may be used in diagnostic assays which detect proteins. For example, an abortive promoter cassette linker can be made with a protein modifier group attached, and used to link the APC to a protein. Suitable proteins include, but are not limited to, antibodies, binding proteins, avidin, signalling molecules, protein A, and the like. In some embodiments, the APC is linked to an antibody specific for a target protein molecule of interest. The antibody binds to the target protein molecule of interest, and the attached APC is used to produce multiple reiterative oligonucleotide transcripts which are then detected with mass spectroscopy. By linking an APC to an antibody or other protein specific for a target molecule, this method can also be used with nonproteinaceous target molecules of interest.

Disease Detection

The present invention is useful for detecting diseases in animal by monitoring the status of DNA methylation, genetic mutations, mRNA expression patterns, and protein expression patterns. The invention can be used to detect, diagnose, and monitor diseases, and/or disorders associated with the aberrant expression and/or activity of a polypeptide or polynucleotide. The invention provides for the detection of the aberrant expression of a polypeptide or polynucleotide, the presence of mutations, and changes in methylation status of DNA. The diagnostic assays of the invention can be used for the diagnosis and prognosis of any disease, including but not limited to Alzheimer disease, muscular dystrophy, cancer, breast cancer, colon cancer, cystic fibrosis, fragile X syndrome, hemophilia A and B, Kennedy disease, ovarian cancer, lung cancer, prostate cancer, retinoblastoma, myotonic dystrophy, Tay Sachs disease, Wilson disease, and Williams disease. These assays are believed to be particularly useful for the diagnosis and prognosis of all types of cancer.

Kits

In some embodiments, the invention is a kit. In one embodiment, the kit comprises a vial containing an abortive promoter cassette containing a linker for coupling the APC to a molecule (such as a nucleic acid or antibody) with specificity for a target molecule of interest. In another embodiment, the kit contains a dendrimer, APCs and coupling reagents. In another embodiment, the dendrimer is coupled to APCs. Other vials contain reagents suitable for effecting the aforesaid coupling. The kit may also contain polymerase, initiators suitable for the APC, and nucleotides suitable for abortive transcription.

Detection Devices

The abortive transcription technology may be incorporated in a device such that one can rapidly and in portable manner detect the presence of a target molecule of interest. Such a device may be a microfluidic device incorporating reaction components such that a sample can be added into the device at one port, with oligonucleotide transcripts obtained from a second port, and then fed into a mass spectrometer. In some embodiments, a single device may couple sample collection, abortive transcription reaction and mass spectrometric detection in a single device.

### EXAMPLES

The following examples are provided for purposes of illustration only and not of limitation. Those of skill in the art will readily recognize a variety of non-critical parameters which could be changed or modified to yield essentially similar results. For example, the individual actions recited may not necessarily be limited to the order presented herein.

### Example 1

RNA Primer-Initiated Abortive Transcription with an RNA Polymerase

Reaction conditions have been optimized for abortive transcription initiation. The components and concentrations of Buffer T favor abortive transcription initiation. Buffer T is comprised of: 20 mM Tris-HCl pH 7.9, 5 MM MgCl₂, 5 mM beta-mercaptoethanol, 2.8% (v/v) glycerol. Primers are either ribonucleoside-triphosphates (NTPs) or dinucleotides ranging in concentration from 0.2-1.3 mM. Final NTP concentrations range from 0.2-1.3 mM. The high ends of the concentration ranges are designed for preparative abortive transcription. The template DNA concentration is less than 2 μM in terms of phosphate. E. coli RNA polymerase is added to a final concentration of between 15 nM and 400 nM. Either holoenzyme or core can be used with a single-stranded template DNA. Yeast inorganic pyrophosphatase is added to 1 unit/ml in preparative reactions to prevent the accumulation of pyrophosphate. At high concentrations pyrophosphate can reverse the synthesis reaction causing RNA polymerase to regenerate NTPs at the expense of the RNA products. One unit of pyrophosphatase is defined as the amount of enzyme to liberate 1.0 μM of inorganic orthophosphate per min. at 25° C. and pH 7.2. Reactions are incubated at 37° C. for up to 72 hours for preparative reactions. These conditions are representative; for specific templates, optimization of particular components and concentrations may enhance the efficiency of abortive initiation.

Three different initiators are used in this example: (1) TAMARA-ApG; (2) Biotin ApG; and (3) ApG. The target nucleic acid template is denatured by boiling for 5 minutes at 95° C. and immediately placing on ice. Each reaction is prepared as follows:
5.0 μl 1X Buffer T
2.5 μl of a-32P-UTP
14.3 μl ddH20
1 μl of E. coli RNA polymerase (1 U/μl)
100 ng (2 μl) of template DNA
10 nmoles ( 1.2 μl) of initiator
22.8 μl of reaction buffer

Incubated at 37° C. for 12-16 hours. The samples are subjected to mass spectrometry and the resultant peaks which correspond to the molecular weights of TAMARA-APG; Biotin ApG; ApG, and breakdown products thereof, are used to determine the presence of the target molecule. The existence of the species may also be confirmed with thin layer chromatography using standard methods known in the art to determine the extent of incorporation of UTP in the third position.

### Example 2

Abortive Initiation Reaction with a Labeled Terminator

Abortive transcription initiation reactions are performed with a labeled initiator and/or a labeled terminator. The following reaction conditions are used to incorporate a labeled terminator:

5 μl 1 X Buffer T

3 μl 100 ng denatured DNA template (pBR322)

13.5 μl dd H₂O

1 μl E. coli RNA polymerase

1.2 μl dinucleotide initiator ApG

1.5 μl of 7mM SF-UTP

Mixtures are incubated at 37° C. for 16 hours in a temperature controlled microtitre plate reader. Thin layer chromatography is performed using standard methods known in the art to demonstrate that the labeled trinucleotide ApGpU was generated. The sample is examined with mass spectrometry to determine that trinucleotide ApGpU was produced. As predicted in Table 1, mass spectrometry should be readily able to distinguish between trinucleotide species.

Detection via mass spectrometry allows simultaneous detection of multiple target molecules or target sequences without the need for reporter tagged ribonucleotide analogs. Table 1(a) shows 20 trinucleotides with different MW. Table 1(b) shows the same 20 trinucleotides sorted by molecular weight. Further multiplexing may also be achieved by using reporter-tagged initial and/or terminator nucleotides.

TABLE 1(a)
Detection of trinucleotide abortive transcripts via Mass
Spectrometry.

| | Nucleotide Position | | | |
|---|---|---|---|---|
| Trinucleotide | 1 | 2 | 3 | MW |
| ApApA | 267 | 347 | 347 | 961 |
| ApApC | 267 | 347 | 323 | 937 |
| ApApG | 267 | 347 | 363 | 977 |
| ApApU | 267 | 347 | 324 | 938 |
| ApCpG | 267 | 323 | 363 | 953 |
| ApGpU | 267 | 363 | 324 | 954 |
| ApCpU | 267 | 323 | 324 | 914 |
| CpCpC | 243 | 323 | 323 | 889 |
| CpCpA | 243 | 323 | 347 | 913 |
| CpCpG | 243 | 323 | 363 | 929 |
| CpCpU | 243 | 323 | 324 | 890 |
| CpGpU | 243 | 363 | 324 | 930 |
| GpGpG | 283 | 363 | 363 | 1009 |
| GpGpA | 283 | 363 | 347 | 993 |
| GpGpC | 283 | 363 | 323 | 969 |
| GpGpU | 283 | 363 | 324 | 970 |
| UpUpU | 244 | 324 | 324 | 892 |
| UpUpA | 244 | 324 | 347 | 915 |
| UpUpC | 244 | 324 | 323 | 891 |
| UpUpG | 244 | 324 | 363 | 931 |

TABLE 1(b)
Detection of trinucleotide abortive transcripts via Mass
Spectrometry: Trinucleotides ordered according to molecular mass

| | Nucleotide Position | | | |
|---|---|---|---|---|
| Trinucleotides | 1 | 2 | 3 | MW |
| CpCpC | 243 | 323 | 323 | 889 |
| CpCpU | 243 | 323 | 324 | 890 |
| UpUpC | 244 | 324 | 323 | 891 |
| UpUpU | 244 | 324 | 324 | 892 |
| CpCpA | 243 | 323 | 347 | 913 |
| ApCpU | 267 | 323 | 324 | 914 |
| UpUpA | 244 | 324 | 347 | 915 |
| CpCpG | 243 | 323 | 363 | 929 |
| CpGpU | 243 | 363 | 324 | 930 |
| UpUpG | 244 | 324 | 363 | 931 |
| ApApC | 267 | 347 | 323 | 937 |
| ApApU | 267 | 347 | 324 | 938 |
| ApCpG | 267 | 323 | 363 | 953 |
| ApGpU | 267 | 363 | 324 | 954 |
| ApApA | 267 | 347 | 347 | 961 |
| GpGpC | 283 | 363 | 323 | 969 |
| GpGpU | 283 | 363 | 324 | 970 |
| ApApG | 267 | 347 | 363 | 977 |
| GpGpA | 283 | 363 | 347 | 993 |
| GpGpG | 283 | 363 | 363 | 1009 |

### Example 3

RNA Primer-Initiated Abortive Transcription With E. coli RNA Polymerase Holoenzyme.

E. coli RNA polymerase holoenzyme can initiate transcription from single-stranded DNA molecules lacking a promoter sequence. Denatured poly[dG-dC] (10 μg/25 μl reaction) is transcribed with E. coli RNA polymerase holoenzyme (1.9 pmoles/reaction). Abortive transcription is initiated with the dinucleotide GpC. GTP is the sole nucleoside-triphosphate available to elongate the primer. The other nucleoside-triphosphate encoded by the template strand (CTP) is omitted. Mass spectrometry is performed, indicating that the presence of the trinucleotide product GpCpG is dependent on GTP concentration and that the detectable product is of one size, suggesting that omission of CTP effectively terminated transcription after the formation of the trinucleotide product.

E. coli RNA polymerase holoenzyme will have a strong preference for bubble complex substrates over template strands that lacked a paritally complementary non-template partner. The relative transcriptional activities by E. coli RNA polymerase holoenzyme with a DNA bubble complex verses the corresponding single template strand are determined, showing that the RNA polymerase exhibits a 70-fold higher levels of activity with Bubble complex 1 than when it is provided with an equivalent molar amount of the template strand alone. Similar results will be obtained in experiments examining the preference of T7 RNA polymerase for bubble complex DNA.

RNA polymerases with diverse promoter recognition properties have been shown to use bubble complex 1 as a substrate for abortive transcription.

In the present experiment Bubble complex 1 is incubated with E. coli holoenzyme, E. coli core RNA polymerase, phage T7 and phage SP6 RNA polymerases. The reaction buffer for E. coli holoenzyme and E. coli core polymerases include 150 mM Na-acetate. Na-acetate is omitted from the T7 and SP6 reactions because high salt concentrations inhibit these enzymes. All reactions contain 20 mM HEPES pH 8 buffer, 10 mM MgCl12 and 2 mM DTT. The initiator ApA and UTP is each provided at 1 mM in all of the reactions. It is expected that E. coli holoenzyme will produce about 2-fold more product than E. coli core polymerase and about 10-fold more product per polymerase than the T7 and SP6 polymerases.

Sensitivity of assays based on primer-initiated abortive transcription with radioactive precursors and autoradiographic detection.

The sensitivity of a detection assay based on a primer-initiated abortive transcription reaction may be determined by defining the minimal amount of Bubble complex 1 that could produce a detectable signal. For example, a series of abortive transcription reactions may be performed with decreasing amounts of Bubble complex 1 (10 femptomoles −1 zeptomole/25 μl reaction). Transcription is initiated with ApA and radioactive UTP. UTP is the only nucleoside triphosphate included in the reactions in order to limit the product to the trinucleotide ApApU. Using mass spectrometry a time-course for each transcription reaction is conducted. After a 3 hour RNA polymerase abortive transcription reaction, signal from 10 femptomoles of bubble complex will be clearly detectable and a faint signal from 1 femptomole of bubble complex is discernable via mass spectrometry. An ApApU signal from 100 attomoles of Bubble complex 1 is detectable after 24 hours of transcription.

### EXAMPLE 4

Protocol for Quantitation Of Abortive Transcription Products (Abscripts) By Reversed-Phase HPLC/Electrospray Ionization Mass Spectrometry

Detection of abscripts by electrospray ionization mass spectrometry (ESI-MS) requires separation of the abscripts from other salts and ions present in the reaction solution. Without this separation, ion suppression prevents detection of the abscripts. This protocol describes the chromatographic and ESI-MS settings that were used for detection and quantitation of abscripts generated from a standard abscription reaction. The protocol includes examples of standard and sample preparations for use with normal solution abscription reactions, and an approach to processing the generated data for quantitation of the abscripts generated in an abscription reaction. One of ordinary skill in the art can further modify and adapt the protocol for use in different conditions.

### HPLC

Waters Alliance 2795 Separations Module controlled with MassLynx software on a PC.

Waters Atlantis C₁₈ column: 2.1×30 mm, 300 μm particle size

HPLC grade acetonitrile and water

Gradient conditions:

Flow rate = 0.4 mL/min.
Solvent A = water
Solvent B = acetonitrile

| 0.00 min. | 0% B | 100% A | |
|---|---|---|---|
| 3.00 min. | 7.5% B | 92.5% A | linear ramp |
| 3.10 min. | 80% B | 20% A | linear ramp |
| 4.00 | 80% B | 20% A | hold |

1 min. re-equilibration at starting conditions before next run

Injection volume: 10 μL

ESI-MS

Waters/Micromass LCT Premier ESI-MS w/Lockspray attachment run in negative ion V-mode, controlled with MassLynx software on a PC, using centroid data collection. High purity N₂ gas supplied by a nitrogen generator (Peak Scientific, NM30LA) Leucine enkephalin (1 ng/mL) for use as the lockspray reference mass standard. Effluent from HPLC column is introduced unsplit into MS source

ESI-MS source settings:

| | |
|---|---|
| Capillary voltage: | 2700 |
| Sample cone: | 50 |
| Desolvation gas temp.: | 300° C. |
| Source temp.: | 100° C. |
| Desolvation gas flow: | 400 |
| Cone gas flow: | 0 |
| Lockspray flow rate: | 4 μL/min. |

ESI-MS ion guide settings:

| | |
|---|---|
| Ion guide 1 | 5.0 |
| Aperture 1 | 5.0 |
| Ion Energy | 105.0 |
| Aperture 2 | 4.0 |
| Hexapole DC | 6.0 |
| Aperture 3 | 4.0 |
| Acceleration | 200.0 |
| Y Focus | 3.0 |
| Steering | 0.2 |
| Tube Lens | 200.0 |
| Attenuated Z-focus | 500.0 |
| Normal Z Focus | 65.0 |

ESI-MS pusher, flight tube, and detector settings

| | |
|---|---|
| TOF Flight tube | 5630.0 |
| Reflectron | 1780.0 |
| Pusher | 820.0 |
| Pusher offset | −0.2 |
| Puller | 720.0 |
| Puller offset | 0.0 |
| MCP detector | 2700.0 |
| Pusher frequency | 29,411.76 Hz |

Standard and Sample Preparations

IDT duplex buffer:

30 mM HEPES

100 mM potassium acetate

pH 7.5

RNA trinucleotide standards purchased from Dharmacon, Inc. at 5 mM in duplex buffer.

5X bubble buffer
100 mM HEPES, pH 8
50 mM MgCl₂
0.5 mM EDTA
40 mM Spermidine
5 mM DTT
750 mM Sodium acetate
150 μg/mL acetylated BSA
14% glycerol
in nuclease-free water

Ribomaker polymerase (883 fmol/μL)

APC bubble at 50 fmol/μL

appropriate NpN initiator at 5 mM in duplex buffer

appropriate NTP at 5 mM in duplex buffer

Standard Preparation:

°μL solutions were prepared with trinucleotide concentrations of 0.5, 1, 5, 10, 50, and 100 μM in a matrix of 1X bubble buffer, 70.6 fmol/μL Ribomaker polymerase, 8 fmol/μL APC bubble, 1 mM appropriate NTP, and 1 mM appropriate NpN initiator. Standard solutions were chilled on ice or at 4° to prevent any abscription from occurring. Alternatively, one may also use APC bubble that will not generate abscripts from the initiator/NTP combination in solution.

Sample Prepration:

Abscription protocol was performed as detailed elsewhere herein. Standards and samples to be evaluated were diluted 1/10 in HPLC grade water and run using HPLC/ESI-MS conditions listed above.

Data Processing

Chromatograms were generated using the appropriate M/Z value for the abscript being evaluated. Use of the doubly charged signal generally will give higher sensitivity. The abscript and initiators have a retention time of 2-3 min. The chromatograms were integrated using default integration parameters. A standard curve was generated fitting the data from the standard preparations to a straight line, which was used to calculate the concentration of abscript generated in the abscription reaction.

### EXAMPLE 5

Mass Spectrometry Detection of AAG and AUG controls

Chemically synthesized AAG and AUG trinucleotides were detected using the electrospray ionization mass spectrometer (ESI-MS).

Materials:

AAG and AUG trinucleotides

HPLC grade acetonitrile (EM science) and water (Fisher).

Triethylamine acetate, pH 7

Methods:

Samples were introduced into the MS by direct infusion at 10 μL/min and evaluated under negative ion mode with V-optics. Source conditions: 2500 V capillary, 35 V cone, 150° desolvation temperature, 80° source, 550 L/min desolvation flow. A 10 μL sample was evaluation, and thus data was acquired for 1 min. in a series of 1 sec. scans with a 0.1 sec. interval between scans.

Results

Mass spectra of 50 pmols of the AAG and AUG trinucleotides are shown in FIG. 18. The parent ions were observed at 936.9 Da and 914.0 Da for AAG and AUG, and actual MW are predicted to be 941.6 and 918.6. The signal from the parent ions in both cases was very weak, but the signal from the doubly charged ion was stronger. These signals occur at half the mass of the parent ion at 468.3 and 456.8 Da, respectiviely. FIG. 19 shows the spectra for AAG at levels of 50, 5, and 0.5 pmol. AAG was detectable at 0.5 pmol.

### EXAMPLE 6

Quantitative evaluation of trinucleotides by RP-HPLC/MS

Reverse-phase HPLC/mass spectrometry was used to detect and quantify (1) the trinucleotide AAG in spiked samples of AAG suspended in abscription reaction mixtures, and (2) as AAG generated by actual abscription reactions.

Experimental Methods

Materials:

Atlantis C₁₈ column: 2.1×30 mm, 300 μm particle size

HPLC grade acetonitrile (EM science) and water (Fisher).

AAG trinucleotide

bubble 22 (generates AAG abscript)

Methods:

Abscription

Using stock bubble concentrations of 50, 35, 20, and 5 fmol/μL, abscription reactions were run following the standard protocol.

Abscription Mixture:

| | |
|---|---|
| Water | 2 μL |
| Bubble buffer (5X concentrate) | 2.5 μL |
| Stock bubble solution | 2 μL |
| Abscriptase (883 fmol/μL) | 1 μL |
| ApA (5 mM) | 2.5 μL |
| GTP (5 mM) | 2.5 μL |

The abscription reactions were run at 45° C. for 30 min., then diluted 20 μL to 200 μL with water before running on the LC/MS.

RP-HPLC/MS Conditions

Mobile Phase:
component A: water
component B: acetonitrile

Gradient Conditions:

| | |
|---|---|
| 0 min. | 100% A |
| 1.4 min. | linear ramp to 70% B |
| 7.0 min. | hold at 70% B |
| 12 min. | immediate switch after 7 min. to 100% A, |
| | hold for 5 min. |
| Flow rate: | 0.2 mL/min |
| Injection volume: | 10 μL |

MS Conditions:
capillary voltage: 2500
source voltage: 35
desolvation temp.: 180° C.
source temp.: 80° C.
desolvation gas flow rate: 750 L/hr
run in V-mode
   Samples Run:

AAG solutions with concentrations of 100, 70, 40, and 10 μM were prepared in water. These samples span a range of 10% to 1% conversion that is commonly observed in abscription reactions. AAG-spiked samples were prepared at the same concentrations above but in a mixture containing the normal concentrations of abscription reagents. Abscription reactions were set-up using bubble stock concentrations of 50, 35, 20, and 5 fmol/μL. A negative control was also run, containing template strand only. All samples were diluted 20 μL to 200 μL with water before running.

Results

FIG. 20A shows the mass spectra generated from two abscription reactions spiked with 10 and 100 μM AAG. In both cases the dominant signal is at 593 Da, which is from the 1 mM ApA present in the reaction mixture.

The HPLC conditions used result in near co-elution of AAG and ApA as shown in FIG. 20B. FIGS. 20A and 20B show two different representations of the data that are used for quantitating the amount of abscript generated from a reaction. FIG. 20A presents the amount of abscript in the form of a signal intensity arising from individual mass spectra summed over breadth of the chromatographic peak shown in FIG. 20B.

Table 2 demonstrates the relationship between different known amounts of AAG and the resulting signal. Samples were prepared both in water alone, and in a solution matrix identical to the abscription reaction. Three quantitative approaches were taken to evaluate the data:

1. MS intensity: the individual scans that comprise the chromatographic peak, as shown in FIG. 20B, were summed together resulting in a single mass spectrum, as shown in FIG. 20A, whose intensity should correlate with the amount of abscript.

2. Peak height and 3. Peak Area: following standard chromatographic approaches, the chromatograms of the AAG signal, like that in FIG. 20B, can be quantified by either peak height or peak area.

TABLE 2
Signals produced from solutions with known amounts of trinucleotide.
The last row in the table is the coefficient of determination for the least-
squares fitted line for the signal of interest plotted as a function of AAG conc.

| | in water | | | in abscription reaction | | |
|---|---|---|---|---|---|---|
| AAG Conc. | MS | Peak | | MS | | |
| (uM) | Intensity | Ht | Peak Area | Intensity | Peak Ht | Peak Area |
| 10 | 1245 | 3749 | 499.62 | 1359 | 4265 | 519.077 |
| 40 | 4121 | 12470 | 1693.787 | 4530 | 14119 | 1799.24 |
| 70 | 6545 | 18785 | 2627.618 | 7629 | 22735 | 3084.891 |
| 100 | 11442 | 29863 | 4601.606 | 10652 | 30113 | 4239.686 |
| R² | 0.974 | 0.989 | 0.974 | 1 | 0.996 | 0.999 |

Table 2 demonstrates that all three quantitative approaches show a relatively linear response to the abscript, in either water or the abscription reaction matrix. The level of performance indicates that a calibration curve generated from spiked solutions such as those used to generate the data in Table 2 may be accurately used to determine the concentration of abscript in an unknown sample. Additional data points and duplication may also be used to increase the accuracy of the calibration curve.

Following this approach, calibration curves for the three quantitative measures of MS intensity, peak height, and peak area generated from the data in Table 2 were used to calculate the concentration of abscript in new samples. Reactions using several known concentrations of APC resulted in several solutions containing unknown concentrations of abscript. These solutions were evaluated using the LC/MS method and the three quantitative measures described above. The resulting concentration values were used, along with the known concentrations of APC, to calculate the turnover rates for the APC tested. The results are presented in Table 3, indicating that increased amounts of APC correlates with increased turnover. However, for samples that fell outside the calibration curve, extrapolation from the calibration curve did not always results in consistent calculations of the turnover rate (see, e.g 5 fmol/μL APC concentration). This problem is remedied by a greater number and range of data points in the calibration curve.

TABLE 3
Detection and calculation of turnover rates generated by abscription
reactions using various amounts of APC. Data from the spiked reactions
were used as a standard curve to quantitate the amount of AAG
produced, and these amounts were used in the turnover calculations.

| | | | | Calculated | | |
|---|---|---|---|---|---|---|
| bubble 22 | | | | Turnover Rates | | |
| Conc. | MS | | | MS | Peak | Peak |
| (fmol/uL) | Intensity | Peak Ht | Peak Area | Intensity | Ht | Area |
| 5 | 467 | 1251 | 153.196 | 42 | −110 | 25 |
| 20 | 1505 | 4711 | 574.306 | 115 | 98 | 112 |
| 35 | 2934 | 9571 | 1204.808 | 148 | 157 | 154 |
| 50 | 4518 | 14627 | 1880.771 | 168 | 183 | 176 |
| template | 119 | 212 | 31.295 | −10 | −26 | −10 |
| R² | 0.992 | 0.993 | 0.990 | | | |

### EXAMPLE 7

Multiplex Abortive Transcription Reaction

The ability to simultaneously conduct multiple abortive transcription (Abscription) reactions at one time, i.e. multiplexing, was demonstrated on a reaction mix containing two APC. Two APCs shown in FIG. 21, Bubble 4 and APC UC2361, were selected for this purpose. Both abortive promoter cassettes were constructed with the same target site probe (TSP), A192, but with different template strands (TEM), Al 97 or A56. The initiator for bubble 4 is ApA and for UC2361 it is UpC and the incorporating nucleotide is GTP. The expected abscription product from Bubble 4 is AAG and from UC2361 is UCG which are distinguishable by TLC and Mass Spectrometry.

The reaction components are listed in Table 4. The volume given in each column is for two reactions so that duplicates can be run. Each reaction was performed in duplicate. The final volume per reaction was 12.5 μl. The reaction components were assembled in a PCR tube in the order listed in Table 4. The reaction was incubated at 45° C. for 30 minutes and 2 μl of the abscription products were spotted on a Silica gel (Al-backed) TLC plate. The TLC was developed in a solvent mix of 6:3:1 of Isopropanol:NH₄OH:H₂O and then dried. The TLC plate was then exposed to phosphor imager screen for 20 minutes and the turnover number (transcript/template/minute) was calculated from the intensities of the spots. FIG. 22 shows the abscription products from the mixed APC experiment by TLC. Lanes 1-3 have bubble 4 (100 fmol final) and initiated with ApA (lane 1), UpC (lane 2), ApA+UpC (lane 3). Lanes 4-6 have equimolar concentration of bubble 4(100 fmol final) and APCUC2361(100 fmol final) and they were initiated with ApA in lane 4, with UpC in lane 5 and with both ApA+UpC in lane 6. Lanes 7-9 have APCUC2361(100 fmol final) and initiated with UpC (lane 7), ApA (lane 8) and ApA+UpC (lane 9). Lane 10 is the TEMA197 initiated with ApA, lane 11 is the TEM A57 initiated with UpC and lane 12 is the control for no APC with initiators ApA+UpC. Lane 13 is the control for the α³²P-GTP. The initiator concentrations were 1 mM final for each.

In this experiment, each abscription reaction used different templates and different initiators but both incorporate GTP. The results demonstrate that, when two different APC are present in the same reaction mixture, there is no inhibition of one abscription reaction due to the presence of the other APC. When the APCs are mixed together there was no appreciable decrease in total turnover, even though the same GTP nucleotide is incorporated (lanes 4-6). Incorrect incorporation, that might indicate the wrong inititor interacting with an APC, was not observed (lanes 2, 3, 6, 8 and 9).

TABLE 4
The reaction components for the mixed APC reactions

| | | | | | | | 6. | | | | | | 12. | | |
|---|---|---|---|---|---|---|---|---|---|---|---|---|---|---|---|
| | | | | 3. | 4. | 5. | Bub4 | | | 9. | 10. | 11. | No | | |
| | | 1. | 2. | Bub4 | Bub4 | Bub4 | UC | 7. | 8. | UC | TEM | TEM | APC | 13. | |
| | Initial | Bub4 | Bub4 | ApA | UC | UC | ApA | UC | UC | ApA | A197 | A56 | ApA | □³²P- | Final |
| Components | con. | ApA | UpC | UpC | ApA | UpC | UpC | UpC | ApA | UpC | ApA | UpC | UpC | GTP | con |
| H₂O | | 6.5 | 6.5 | 4 | 2.5 | 2.5 | — | 6.5 | 6.5 | 4 | 6.5 | 6.5 | 13 | 17.5 | |
| Bubble | 5× | 5 | 5 | 5 | 5 | 5 | 5 | 5 | 5 | 5 | 5 | 5 | 5 | 5 | 1× |
| Buffer | | | | | | | | | | | | | | | |
| Bubble 4 | 50 fmol/μl | 4 | 4 | 4 | 4 | 4 | 4 | — | — | — | — | — | — | — | 8 fmol/μl |
| UC2361 | 50 fmol/μl | — | — | — | 4 | 4 | 4 | 4 | 4 | 4 | — | — | — | — | 8 fmol/μl |
| TEM A197 | 50 fmol/μl | — | — | — | — | — | — | — | — | — | 4 | — | — | — | 8 fmol/μl |
| TEM A56 | 50 fmol/μl | — | — | — | — | — | — | — | — | — | — | 4 | — | — | 8 fmol/μl |
| E. coli holo | 883 fmol/μl | 2 | 2 | 2 | 2 | 2 | 2 | 2 | 2 | 2 | 2 | 2 | 2 | — | 70 fmol/μl |
| RNAP | | | | | | | | | | | | | | | |
| ApA | 10 mM | 2.5 | — | 2.5 | 2.5 | — | 2.5 | — | 2.5 | 2.5 | 2.5 | — | 2.5 | — | 1 mM |
| UpC | 10 mM | — | 2.5 | 2.5 | — | 2.5 | 2.5 | 2.5 | — | 2.5 | — | 2.5 | 2.5 | — | |
| α³²P-GTP | 5 mM | 5 | 5 | 5 | 5 | 5 | 5 | 5 | 5 | 5 | 5 | 5 | 5 | 5 | 1 mM |

### EXAMPLE 8

Multiplex Abortive Transcription Reaction

Ten APC were generated that produce different abscripts. FIG. 23 shows a representation of the 10 Abortive Promoter Cassettes made with different combinations of oligonucleotide strands, for use in multiplex abscription reactions.

The annealing of oligonucleotide strands to form the APCs depicted in FIG. 23 was determined by electrophoresis on a 4% agarose gel, as shown in FIG. 24. 4% Agarose gel analysis of the APCs. 5 ul of 1 pmol/μl of the APC was loaded in each lane (1-10). To show the comparison with the single strand, TEM A191 (5 μl of 50 pmol/μl) was loaded in lane 11 and lane 12 has the 50 bp ladder.

Abscription was performed on APCs 4, 12, 24, 25 and 32, and the abscripts run on TLC. Each abscription was done with 100 fmol of APC with 1 mM final concentration of the initiator along with 1 mM final concentration of the NTP as listed in the figure along with 1 μl of the abscriptase. The abscription was done for 60 minutes at 45° C. 2 μl from each reaction was spot on the TLC plate and developed with 6:3:1 of Isopropanol:NH₄OH:H₂O. The LC plate was exposed to phosphor imager screen for 10 minutes.

The abscription from each APCs was detected using radioactivity as shown in FIG. 25. Each APC gave a different abscript, although AAG and AAU could not be readily distinguished by TLC.

### EXAMPLE 9

Abscription-Based Detection of SEB Protein with Anti-SEB Antibody-APC Complex

Staphylococcus aureus enterotoxin B (SEB) was detected using sandwich ELISA and antibodies against SEB that are coupled to Abortive Promoter Cassettes.

Experimental Methods

Coupling

APC 22 (APCAA2361), which generates an AAG abscript, was amine modified, and coupled to antibodies against SEB. After coupling, the mixture of Ab-APC conjugates and unreacted components was separated into fractions using HPLC. Fractions 17-19 and 22-24 were pooled and examined. Pooled samples were concentrated using 30 kDa spin filters. Antibody concentrations in these samples were determined using the micro BCA assay from Pierce.

Sandwich ELISA

SEB-coated plates were blocked with 2% BSA/PBS-Tween 20 for one hour. Antibody conjugate solutions at 10 μg/mL IgG and serial dilutions of ½ are added to the plate in columns 1-11 so that the concentration of antibody in column 1 was 10 μg/mL, column 2 was 5 μg/mL, column 3 was 2.5 μg/mL, etc., and the concentration in column 11 was 9.77 ng/mL. Final volume in each well was 50 μL. Following a 1 hour incubation at room temperature and rocking, the abscription reaction was setup as follows. 10 μL of 883 nM abscriptase in bubble buffer was added to each well followed by 10 μL of abscription reagent containing 2 mM I-ApA, 2 mM T-GTP, 0.1 μCi/μL α-³²p-GTP in bubble buffer. A 20 μL solution control of 1 mM T-030, 0.1 μCi/μL α-³²P-GTP in bubble buffer was placed in an unoccupied well. The plate was incubated at 45° C. with 400 rpm shaking in a thermomixer (Eppendorf) for two hours followed by a cool-down to 4° C. for storage overnight. 2 μL of the reaction solutions and control on aluminum backed silica gel TLC plates (Whatman) and developed. Phosphor screens (Molecular Dynamics) were exposed to the TLC plates for 20 min., imaged using a Phosphoimager:SI (Molcular dynamics) and evaluated and quantified using ImageQuant software (Molecular Dynamics). Calibrators showed the data to be within the linear range of the imager.

Results

The titration curves for the signals generated by the two conjugate pools are shown in FIG. 26. Pool 22-24 corresponds to the peak fractions for the largest conjugate band and pool 17-19 corresponds to a secondary peak that elutes slightly earlier. The conjugates from these two bands have different mobilities when run on non-reducing SDS-PAGE gels (not shown). The different elution times and electrophoretic mobilities suggest that the material eluting in the 17-19 fractions was a conjugate with two APCs bound, while the material from the 22-24 fractions was a conjugate with only a single APC bound. This titration of the abscription reaction shows a distinct difference in the specific abscription activity of the two conjugates and further supports the idea of pool 17-19 having two APCs bound, as against a single APC bound in pool 22-24. A comparative direct ELISA assay did not show a significant difference in binding ability for the conjugates and so the difference in signal observed is primarily due to a difference in abscription ability.

### EXAMPLE 10

Simultaneous detection of 10 trinucleotides by Mass Spectrometry

To demonstrate that at least 10 abscripts can be separately detected with mass spectrometry, 10 chemically synthesized trinucleotides were examined both individually and as a single mixture. FIG. 27 shows the combined chromatogram of all 10 trinucleotides from their individual runs. The embedded chart shows which numbered peak refers to which abscript, as well as the relevant retention time, m/z value, and peak area. FIG. 27 demonstrated that most trinucleotide had a slightly different retention time. However, even if the LC retention time was similar or identical the samples can be evaluated independantly with mass spectrometry. For example, peaks 7 (AUU) and 8 (AAU) show identical retention time, but yielded distinct m/z values.

The ability of mass spectrometry to distinguish different abscripts from a pool of ten abscripts was demonstrated as follows. A mixture of 10 purified trinucleotides were mixed together in equimolar concentrations and analyzed by mass spectrometry. FIG. 28 shows the output of a mixture of trinucleotides detected simultaneously by mass spectroscopy. Each trinucleotides is shown next to its corresponding m/z ratio. 10 ul of a 10 uM solution was analyzed (100 pmol) by Atlantis dC18 3um 2.1 mm X 30 mm column. The MS tune file used was “abscript”.

Table 5 shows, for each abscript, the calculated molecular weight and observed m/z value for the doubly charged ion observed by mass spectrometry.

TABLE 5

| | | |
|---|---|---|
| Abscript | Molecular Weight | m/z Value |

| | | |
|---|---|---|
| AAG | 941.662 | 469.6 |
| AAU | 902.662 | 450.1 |
| ACC | 877.612 | 437.6 |
| AGC | 917.637 | 457.6 |
| AGG | 957.662 | 477.6 |
| AUU | 879.581 | 438.6 |
| UCC | 854.572 | 426.1 |
| UCG | 894.597 | 446.1 |
| UGG | 934.622 | 466.1 |
| CGG | 933.617 | 465.6 |

Accordingly, mass spectrometry is used to distinguish the products of a multiplex reaction containing at least 10 different APCs, and should therefore be able to detect at least 10 different target molecules, or detect a more limited number of targets with greater sensitivity and accuracy.

### EXAMPLE 11

Multiplex Abortive Transcription and Detection with Mass Spectrometry

Two APCs, APC 2-1 and 12-1, shown in FIG. 29, were mixed and the abscription reaction performed for 29 minutes at 45° C., and abscripts were analyzed by LC-MS, all according to previously described protocols. The abscript made by APC 2-1 was AAG (similar to APC 4) and the abscript made by APC 12-1 was AAU. The abscripts AAG and AAU cannot be separated by TLC, as shown in FIG. 25. However, these abscripts can be separated by mass spectrometry. FIG. 31 shows the mass spectrogram from the multiplex reaction with the two APCs, which were readily separated on the basis of m/z value. The data also demonstrates that abortive transcription from one APC was not inhibited by the presence of abortive transcription occuring from the other APC.

FIG. 30 shows a summary of AAU and AAG signals generated from the multiplex abscription reaction compared with control reactions.

Individual ABC 2-1 reaction data is in columns 1 and 2. Individual ABC 12-1 reaction data is in columns 5 and 6. The combined reaction data (reaction was done in duplicate) is in columns 9-12. AAG, AAU and AAG+AAU controls are in columns 13-16.

In the foregoing specification, the invention has been described with reference to specific embodiments. However, it will be appreciated that various modifications and changes can be made without departing from the scope of the present invention as set forth in the claims below. The specification and figures are to be regarded in an illustrative manner, rather than a restrictive one, and all such modifications are intended to be included within the scope of present invention. All cited references are fully incorporated by reference into the specification. While embodiments may comprise various elements or features, it is also considered that such embodiments may also consist essentially of, or consist of, said elements or features.

1. A method for detecting the presence of a target molecule comprising:
(a) synthesizing multiple copies of oligonucleotides through abortive reiterative synthesis on a nucleic acid template; and
(b) determining the presence of said target molecule by mass spectrometry, thereby detecting the presence of said target molecule.

2. The method of claim 1, wherein the molecular mass and abundance of said multiple copies of oligonucleotides is determined.

3. The method of claim 1, wherein said target molecule comprises said nucleic acid template.

4. The method of claim 1, wherein said target molecule is distinct from said nucleic acid template.

5. The method of claim 4, wherein said nucleic acid template is an abortive promoter cassette.

6. The method of claim 1, wherein said nucleic acid comprises multiple abortive promoter cassettes which are linked to at least one second molecule with specificity for the target molecule.

7. The method of claim 6, wherein said multiple abortive promoter cassettes are linked to said second molecule via a dendrimer.

8. The method of claim 1, wherein said abortive promoter cassette is linked to a second molecule with specificity for the target molecule, said second molecule selected from the group consisting of:
(a) a DNA sequence;
(b) a RNA sequence;
(c) a PNA sequence;
(d) an antibody;
(e) a binding protein;
(f) a signalling molecule;
(g) a hapten;
(h) biotin; and
(i) an ion.

9. A method for detecting the presence of a target molecule comprising:
(a) incubating a template polynucleotide with an initiator and a polymerase;
(b) synthesizing multiple oligonucleotides from said template polynucleotide, wherein said initiator is extended until the transcript is terminated, causing multiple reiterative oligonucleotide transcripts to be synthesized; and
(c) determining the presence of said target molecule by mass spectrometry.

10. The method of claim 9, wherein said multiple reiterative oligonucleotide transcripts further comprise a modified nucleotide.

11. The method of claim 9, further comprising incubating said target polynucleotide with a target site probe.

12. The method of claim 11, wherein said target site probe is of a size selected from the group consisting of: about 20 to about 50 nucleotides; about 51 to about 75 nucleotides; about 75 to about 100 nucleotides; and greater than 100 nucleotides.

13. The method of claim 1, wherein said target polynucleotide is DNA, and said multiple reiterative oligonucleotide transcripts comprise RNA.

14. The method of claim 1, wherein said target molecule is selected from the group consisting of:
(a) a protein;
(b) nucleic acid
(c) RNA;
(d) DNA;
(e) a Carbohydrate;
(f) a Lipid;
(g) an Antigen;
(h) a Hapten; and
(i) an Ion.

15. The method of claim 1, wherein a pathogen is detected.

16. The method of claim 1, wherein said molecule is from an organism selected from the ground consisting: HIV-1; HIV-2; HIV-LP; polio virus; hepatitis A virus; entero virus; human coxsackie viruses, rhinovirus; echovirus; Calciviridae; Togaviridae; equine encephalitis virus; rubella virus; Flaviridae; dengue virus; encephalitis virus, yellow fever virus; Coronaviridae; SARS; Rhabdoviridae; vesicular stomatitis virus; rabies virus; Filoviridae; ebola virus; Paramyxoviridae; parainfluenza virus, mumps virus, measles virus, respiratory syncytial virus; Orthomyxoviridae; influenza virus; Bunyaviridae; Hantaan virus; bunya virus, phlebovirus; Nairo virus; Arenaviridae; Reoviridae; reovirus; orbivirus; rotavirus; Birnaviridae; Hepadnaviridae; Hepatitis B virus; Parvoviridae; Papovaviridae; papilloma virus; polyoma virus; Adenoviridae; Herpesviridae; HSV 1;
HSV 2; varicella zoster virus; cytomegalovirus (CMV); Poxviridae;
variola virus; vaccinia virus; Iridoviridae; African swine fever virus;
Hepatitis C; Norwalk virus Helicobacter pylori, Borelia burgdorferi, Legionella pneumophilia, M tuberculosis, M avium, M intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans group, Streptococcus faecalis, Streptococcus bovis, anaerobic Streptococcus spp., Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Actinomyces israelli; Cryptococcus neoformans, Histoplasma capsulatum, Coccidioides immitis, Blastomyces dermatitidis, Chlamydia trachomatis, Candida albicans; Plasmodium falciparum; and Toxoplasma gondii.

17. The method of claim 1, wherein a cancer is detected.

18. The method of claim 1, wherein DNA methylation is detected.

19. The method of claim 1, wherein a mutation is detected.

20. The method claim 1, wherein a disease is detected.

21. The method of claim 1 wherein RNA expression is quantified.

22. The method of claim 1, wherein said mass spectrometry consists of any one of the methods selected from:
(a) fast atomic bombardment (FAB) mass spectrometry;
(b) plasma desorption (PD) mass spectrometry;
(c) electrospray/ionspray (ES) mass spectrometry;
(d) matrix-assisted laser desorption/ionization (MALDI) mass spectrometry; and
(e) matrix-assisted laser desorption/ionization time of flight analysis (MALDI-TOF).

23. A composition comprising a dendrimer to which is linked one or more abortive promoter cassettes.

24. The composition of claim 23, comprising a dendrimer to which is linked multiple abortive promoter cassettes.

25. The composition of claim 24, wherein said multiple abortive promoter cassettes linked to said dendrimer are identical.

26. The composition of claim 24, wherein said multiple abortive promoter cassettes linked to said dendrimer are nonidentical.

27. The composition of claim 23, further linked to a second molecule with specificity for a target molecule of interest.

28. The composition of claim 27, wherein said second molecule selected from the group consisting of:
(a) a DNA sequence;
(b) a RNA sequence;
(c) a PNA sequence;
(d) an antibody;
(e) a binding protein;
(f) a signalling molecule;
(g) a hapten;
(h) biotin; and
(i) an ion.

29. The composition of claim 28, wherein said second molecule is an antibody

30. The composition of claim 28, wherein said second molecule is a DNA sequence.

31. A kit, containing the composition of claim 23, and one or more buffers, and one or more coupling reagents.

32. A method for detecting the presence of a target molecule comprising:
(a) synthesizing multiple copies of oligonucleotides through abortive reiterative synthesis on a nucleic acid template;
(b) introducing said multiple copies of abortive reiteratively synthesized transcripts into a signal amplification cascade; and
(c) determining the presence of the signal from said signal cascade, thereby detecting the presence of said target molecule.

33. The method of claim 32, wherein said signal amplification cascade is selected from the group consisting of:
(a) PCR;
(b) Abortive transcription;
(c) FRET; and
(d) Enzymatic amplification.

34. A method for detecting the presence of one or more target molecules comprising:
(a) simultaneously performing two or more different syntheses of multiple copies of oligonucleotides through abortive reiterative synthesis on a nucleic acid template; and
(b) determining the presence of said target molecule thereby detecting the presence of two or more target molecules.

35. An abortive promoter cassette comprising:
(a) an upstream arm length greater than 13 nt and a downstream arm length greater than 13 nt;
(b) a bubble segment length of 11 to 14 nt comprising
(i) a nontemplate strand comprising the sequence: 5′ TANNNTN₅₋₈ and
(ii) a template strand selected from the group consisting of
A) 3′C(C or G or A)N₉₋₁₃;
B) A(C or G or A)N₉₋₁₃;
C) T(C or G or A)N₉₋₁₃; and
D) G(C or G of A)N₉₋₁₃;
wherein abortive transcription proceeds off said abortive promoter cassette.

36. The abortive promoter cassette of claim 35, wherein said nontemplate strand comprises a sequence: 5′ TATAATN₅₋₈.

37. The abortive promoter cassette of claim 36, wherein said abortive promoter cassette is selected from the group consisting of:

| | |
|---|---|
| a) | 5′ NNNNNNNNNNNNNTATAATNNNNNNNNNNNNNNNNNNNN |
| | 3′ NNNNNNNNNNNNNCCNNNNNNNNNNNNNNNNNNNNNNNN; |
| b) | 5′ NNNNNNNNNNNNNTATAATNNNNNNNNNNNNNNNNNNNN |
| | 3′ NNNNNNNNNNNNNCGNNNNNNNNNNNNNNNNNNNNNNNN; |
| c) | 5′ NNNNNNNNNNNNNTATAANNNNNNNNNNNNNNNNNNNNN |
| | 3′ NNNNNNNNNNNNNCANNNNNNNNNNNNNNNNNNNNNNNN; |
| d) | 5′ NNNNNNNNNNNNNTATAATNNNNNNNNNNNNNNNNNNNN |
| | 3′ NNNNNNNNNNNNNACNNNNNNNNNNNNNNNNNNNNNNNN; |
| e) | 5′ NNNNNNNNNNNNNATAATNNNNNNNNNNNNNNNNNNNNN |
| | 3′ NNNNNNNNNNNNNAGNNNNNNNNNNNNNNNNNNNNNNNN; |
| and | |
| f) | 5′ NNNNNNNNNNNNNATAATNNNNNNNNNNNNNNNNNNNNN |
| | 3′ NNNNNNNNNNNNNAANNNNNNNNNNNNNNNNNNNNNNNN. |

38. The abortive promoter cassette of claim 35 comprising a target site probe, wherein said target site probe comprises a sequence at least about 80% identical to the sequence selected from the group consisting of:
(a) TSP2361;
(b) TSP2361AAC;
(c) TSP2361AAU;
(d) TSP2361AAA;
(e) TSP2361UCA;
(f) TSP2361CA;
(g) TSP2361bub15;
(h) the target site probe of bubble 4;
(i) TSP2-1; and
(j) TSP12-1.

39. The abortive promoter cassette of claim 38, comprising a target site probe wherein said target site probe comprises a sequence selected from the group consisting of:
(a) TSP2361;
(b) TSP2361AAC;
(c) TSP2361AAU;
(d) TSP2361AAA;
(e) TSP2361UCA;
(f) TSP2361CA;
(g) TSP2361bub15;
(h) the target site probe of bubble 4;
(i) TSP2-1; and
(j) TSP12-1.

40. The abortive promoter cassette of claim 35, comprising a template strand wherein said template strand comprises a sequence at least about 80% identical to the sequence selected from the group consisting of:
(a) TEM2361AA-2;
(b) TEM2361AAC;
(c) TEM2361AA;
(d) TEM2361UCA;
(e) TEMUC2361;
(f) TEM-CpA-2361;
(g) TEM2361AU;
(h) TEM-CpG-2361;
(i) the template strand of bubble 4;
(j) TEM2-1; and
(k) TEM12-1.

41. The abortive promoter cassette of claim 40, comprising a template strand wherein said template strand comprises a sequence selected from the group consisting of:
(a) TEM2361AA-2;
(b) TEM2361AAC;
(c) TEM2361AA;
(d) TEM2361UCA;
(e) TEMUC2361;
(f) TEM-CpA-2361;
(g) TEM2361AU;
(h) TEM-CpG-2361;
(i) the template strand of bubble 4;
(j) TEM2-1; and
(k) TEM12-1.

42. The abortive promoter cassette of claim 35, comprising an abortive promoter cassette selected from the group consisting of:
(a) APC4;
(b) APC11;
(c) APC12;
(d) APC14;
(e) APC13;
(f) APC24;
(g) APC25;
(h) APC30;
(i) APC32;
(j) APC33;
(k) bubble 4;
(l) APC UC2361;
(m) APC2-1; and
(n) APC12-1.

43. An abortive promoter cassette, wherein the sequence of said abortive promoter cassette target site probe and template strand is at least about 80%, about 85%, about 90%, about 95% or about 99% identical to the abortive promoter cassette of claim 42.

44. A method for detecting the presence of a target molecule comprising:
(a) synthesizing multiple copies of oligonucleotides through abortive reiterative synthesis from the abortive promoter cassette of claim 35; and
(b) determining the presence of the signal, thereby detecting the presence of said target molecule.


SEQUENCE LISTING (41 sequences)

SEQ ID NO: 1 - DNA, 39 bp, Artificial Sequence
  APC with possible alternative initiator sites
nnnnnnnnnnnnntataatnnnnnnnnnnnnnnnnnnnn

SEQ ID NO: 2 - DNA, 54 bp, Artificial Sequence
  APC with specific target site probe
ggatacttacagccattatatttagccctactccattccatcccgggttcgtcc

SEQ ID NO: 3 - DNA, 54 bp, Artificial Sequence
  APC with specific target site probe
ggacgaacccgggatggaatggagtattcgccgtgtccatggctgtaagtatcc

SEQ ID NO: 4 - DNA, 30 bp, Artificial Sequence
  Deamination conversion of unmethylated cytosine groups
unnuuuaaannggtuuggagugautnntua

SEQ ID NO: 5 - DNA, 30 bp, Artificial Sequence
  Deamination conversion of unmethylated cytosine groups
uuguuuaaaugggtuuggagugautugtua

SEQ ID NO: 6 - DNA, 30 bp, Artificial Sequence
  Deaminated target DNA
ucguuuaaacgggtuuggagugautcgtua

SEQ ID NO: 7 - DNA, 33 bp, Artificial Sequence
  Target site probe
ntaacgaatcnnnnnnnnnnnngtttaaacgan

SEQ ID NO: 8 - DNA, 14 bp, Artificial Sequence
  Synthetic deoxyribonucleotide polymer of repeating dCpdG
gcgcgcgcgcgcgc

SEQ ID NO: 9 - DNA, 54 bp, Artificial Sequence; identical to SEQ ID NO: 2
  Bubble complex 1 comprising template and non-template strands

SEQ ID NO: 10 - DNA, 54 bp, Artificial Sequence
  Bubble complex 1 comprising template and non-template strands
ggacgaacccgggatggaatggagtnnncgccgtgtccatggctgtaagtatcc

SEQ ID NO: 11 - DNA, 48 bp, Artificial Sequence
  TSP A192
gctgctggaggcgggtataatttagccagcaccgaatagttacggtcg

SEQ ID NO: 12 - DNA, 48 bp, Artificial Sequence
  TEM A197
cgaccgtaactattcggtgcttagggcagcgcccccgcctccagcagc

SEQ ID NO: 13 - DNA, 48 bp, Artificial Sequence
  TEM A56
cgaccgtaactattcggtgcgatgggcagcgcccccgcctccagcagc

SEQ ID NO: 14 - DNA, 48 bp, Artificial Sequence; identical to SEQ ID NO: 11
  TSP 2361

SEQ ID NO: 15 - DNA, 48 bp, Artificial Sequence; identical to SEQ ID NO: 12
  TEM2361AA-2

SEQ ID NO: 16 - DNA, 48 bp, Artificial Sequence
  TSP2361AAC
gctgctggaggcgggtataatttagccaccaccgaatagttacggtcg

SEQ ID NO: 17 - DNA, 48 bp, Artificial Sequence
  TEM2361AAC
cgaccgtaactattcggtggttagggcagcgcccccgcctccagcagc

SEQ ID NO: 18 - DNA, 48 bp, Artificial Sequence
  TSP2361AAU
gctgctggaggcgggtataatttagccatcaccgaatagttacggtcg

SEQ ID NO: 19 - DNA, 48 bp, Artificial Sequence
  TEM2361AA
cgaccgtaactattcggtgattagggcagcgcccccgcctccagcagc

SEQ ID NO: 20 - DNA, 48 bp, Artificial Sequence
  TSP2361AAA
gctgctggaggcgggtataatttagccaacaccgaatagttacggtcg

SEQ ID NO: 21 - DNA, 48 bp, Artificial Sequence
  TEM2361AA-2
cgaccgtaactattcggtgtttagggcagcgcccccgcctccagcagc

SEQ ID NO: 22 - DNA, 48 bp, Artificial Sequence
  TSP2361UCA
gctgctggaggcgggtataatttagcccacaccgaatagttacggtcg

SEQ ID NO: 23 - DNA, 48 bp, Artificial Sequence
  TEM2361UCA
cgaccgtaactattcggtgtgatgggcagcgcccccgcctccagcagc

SEQ ID NO: 24 - DNA, 48 bp, Artificial Sequence; identical to SEQ ID NO: 13
  TEMUC2361

SEQ ID NO: 25 - DNA, 48 bp, Artificial Sequence
  TSP2361CA
gctgctggaggcgggtataatttagctcgcaccgaatagttacggtcg

SEQ ID NO: 26 - DNA, 48 bp, Artificial Sequence
  TEM-CpA-2361
cgaccgtaactattcggtgctgtgggcagcgcccccgcctccagcagc

SEQ ID NO: 27 - DNA, 59 bp, Artificial Sequence
  TSP2361
agtattgacttaaagtctaacctataggatataatagattcacgagagggacacggcga

SEQ ID NO: 28 - DNA, 59 bp, Artificial Sequence
  TEM2361AU
tcgccgtgtccctctcgatggctgtaagtatcctataggttagactttaagtcaatact

SEQ ID NO: 29 - DNA, 48 bp, Artificial Sequence
  TEM2361AU
cgaccgtaactattcggtgctatgggcagcgcccccgcctccagcagc

SEQ ID NO: 30 - DNA, 48 bp, Artificial Sequence
  TSP2361bub15
gctgctggaggcgggtataatttagctcctaccgaatagttacggtcg

SEQ ID NO: 31 - DNA, 48 bp, Artificial Sequence
  TEM-CpG-2361
cgaccgtaactattcggtgccgtgggcagcgcccccgcctccagcagc

SEQ ID NO: 32 - DNA, 48 bp, Artificial Sequence
  TSP 2-1
gctgctggaggcggtataatttagtcaagcaccgaatagttacggtcg

SEQ ID NO: 33 - DNA, 48 bp, Artificial Sequence
  TEM 2-1
cgaccgtaactattcggtgcttagggccgcgcccccgcctccagcagc

SEQ ID NO: 34 - DNA, 48 bp, Artificial Sequence
  TSP 12-1
gctgctggaggcgggtataatttagccaatcaccgaatagttacggtc

SEQ ID NO: 35 - DNA, 48 bp, Artificial Sequence
  TEM 12-1
gaccgtaactattcggtgattacgggccgcgcccccgcctccagcagc

SEQ ID NO: 36 - DNA, 39 bp, Artificial Sequence
  APC with possible alternative initiator sites
nnnnnnnnnnnnnnnnnnnnnnnnccnnnnnnnnnnnnn

SEQ ID NO: 37 - DNA, 39 bp, Artificial Sequence
  APC with possible alternative initiator sites
nnnnnnnnnnnnnnnnnnnnnnnngcnnnnnnnnnnnnn

SEQ ID NO: 38 - DNA, 39 bp, Artificial Sequence
  APC with possible alternative initiator sites
nnnnnnnnnnnnnnnnnnnnnnnnacnnnnnnnnnnnnn

SEQ ID NO: 39 - DNA, 39 bp, Artificial Sequence
  APC with possible alternative initiator sites
nnnnnnnnnnnnnnnnnnnnnnnncannnnnnnnnnnnn

SEQ ID NO: 40 - DNA, 39 bp, Artificial Sequence
  APC with possible alternative initiator sites
nnnnnnnnnnnnnnnnnnnnnnnngannnnnnnnnnnnn

SEQ ID NO: 41 - DNA, 39 bp, Artificial Sequence
  APC with possible alternative initiator sites
nnnnnnnnnnnnnnnnnnnnnnnnaannnnnnnnnnnnn